(19) United States
(12) Patent Application Publication
(10) Pub. No.: US 2010/0111973 A1
(43) Pub. Date: May 6, 2010

(54) METHODS FOR TREATING MICA-RELATED DISORDERS

(76) Inventors: Glenn Dranoff, Lexington, MA (US); Catia Fonseca, Cambridge, MA (US); F. Stephen Hodi, Framingham, MA (US); Masahisa Jinushi, Tokyo (JP)

Correspondence Address: FISH & RICHARDSON PC, P.O. BOX 1022, MINNEAPOLIS, MN, 55440-1022, US

(21) Appl. No.: 12/442,222

(22) PCT Filed: Sep. 24, 2007
(86) PCT No.: PCT/US07/79342

§ 371 Date: Dec. 23, 2009

Related U.S. Application Data
(60) Provisional application No. 60/826,657, filed on Sep. 22, 2006.

(51) Int. Cl. A61K 39/395; G01N 33/53; A61P 35/00

(52) U.S. Cl. 424/172.1; 435/7.1

(57) ABSTRACT

Disclosed herein are materials and methods for treating cancer. In particular, the compositions and methods for treating cancers associated with MICA overexpression are provided.

### TECHNICAL FIELD

This invention relates to the field of oncology, and more particularly to methods and compositions for treating cancer.

### BACKGROUND

Tumor cells express a variety of gene products that provoke innate and adaptive immune recognition. The formation of clinically evident tumors can indicate a failure of host defense. One mechanism that facilitates tumor progression is insufficient tumor antigen presentation. Cancer remains a major cause of human morbidity and mortality and there is a continuing need for therapeutic strategies that consistently stimulate protective immunity.

### SUMMARY

Methods and compositions for cancer therapy are provided. In particular, the methods and compositions described herein stimulate immune-mediated tumor destruction. The present invention is based, in part, on the inventors' observation that induction of high-titer antibodies against the NKG2D ligand, MICA, in cancer patients can provoke a clinical anti-tumor response.

MICA is an MHC class I-related polypeptide detected in some normal gastrointestinal epithelial cells and thymocytes. Double stranded DNA breaks trigger high-level expression of MICA in a broad range of human cancers, including melanoma, lung, breast, kidney, ovarian, prostate, gastric, and colon carcinomas as well as on certain leukemias. MICA is also shed by tumor cells, i.e., released from the cell surface into the surrounding medium, and sera from cancer patients typically contain elevated levels of the soluble form (sMICA). Shed MICA is thought to impair host defense by inducing the internalization of NKD2G molecules on lymphocytes. The inventors have also observed that an increase in sMICA levels is correlated with progression from pre-malignant disorders to malignancy in certain plasma cell cancers, e.g., multiple myeloma. The inventors further observed that the induction of high-titer antibodies against the eneymes involved in MICA shedding, for example, human protein disulfide isomerase (PDI) and the PDI, ERp5, also evoked potent humoral reactions in diverse solid and hematologic malignancy patients.

Accordingly, a method of treating a cancer or a symptom of cancer or cancer progression in a subject is provided. In one embodiment, the method can include administering to the subject an effective amount of a MICA modulating composition. The MICA modulating composition can include an anti-MICA antibody, an anti-protein disulfide isomerase (PDI) antibody or a combination thereof. In some embodiments, the PDI can be ERp5. The MICA modulating composition can include an agent that modulates MICA shedding. In some embodiments, agent that modulates MICA shedding can include a protein disulfide isomerase (PDI) inhibitor. The PDI can be, for example, a human PDI such as ERp5. The method can further include administering one or more tumor cell antigens that elicit an immune response against a tumor. The tumor cell antigens can include autologous tumor cells. The autologous tumor cells can express GM-CSF. In another aspect, the method can further include administering an anti-CTLA-4 antibody to the subject. The anti-CTLA-4 antibody can be administered alone or in combination with the autologous tumor cells.

The cancer can express elevated levels or activity of MICA and can be selected from the group consisting of melanoma, lung cancer, breast cancer, plasma cell cancer, leukemia, lymphoma, ovarian cancer, colon cancer, pancreatic cancer, and prostate cancer. The plasma cell cancer can be multiple myeloma. The symptom of cancer progression can include monogammopathy of undetermined significance (MGUS) or smoldering multiple myeloma. The subject can be a mammal; the mammal can be human.

The anti-MICA antibody can be a monoclonal antibody, a polyclonal antibody, an Fab fragment, a chimeric antibody, a humanized antibody, or a single chain antibody Regardless of the precise molecular form of the anti-MICA antibody, the anti-MICA antibody is a pharmaceutically pure antibody. The anti-MICA antibody can be administered by injection, infusion, or inhalation.

In another embodiment, the methods and compositions further include administering a conventional cancer therapeutic to the subject. The conventional cancer therapeutic can be at least one of chemotherapy, radiation therapy, immunotherapy, hormone ablation or surgery. In one aspect, the conventional cancer therapeutic includes a DNA-damaging agent. Examples of DNA-damaging agents include radiation therapy, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane). In another aspect, the conventional cancer therapeutic can include a proteosome inhibitor; the proteosome inhibitor can be Bortezamib

In another embodiment, the anti-MICA antibody reduces the level of soluble MICA (sMICA) in the subject. The level of sMICA can be the level in serum.

In another embodiment, a method of eliciting an immune response against a cancer in a subject having a MICA-expressing cancer is provided. The method can include; a) identifying a patient having a MICA-expressing cancer; and b) administering an effective amount of an anti-MICA antibody. The method can further include administering a DNA-damaging agent and can be used to treat tumors in which DNA damaging agents are employed to stimulate MICA expression. The method can further include a proteosome inhibitor. Examples of immune responses can include increased NKG2D-dependent cell killing via NK, CD8+ T and NKT cells, increased anti-tumor CD4+ and CD8+ T-lymphocyte toxicity as a consequence of tumor cell cross-presentation, MICA-dependent complement fixation, and MICA-specific antibody-dependent cellular cytotoxicity.

In another embodiment, provided herein is a method of treating cancer comprising administering to a subject in need of treatment an effective amount of an opsonizing agent, where the opsonizing agent binds to MICA. In another aspect, the anti-MICA antibody opsonizes tumor cells.

Also provided is a method for identifying a subject who is a candidate for anti-MICA therapy. The method can include detecting evidence of overexpression of MICA in a sample from the subject as compared to expression of MICA in a control, where the evidence of overexpression of MICA is indicative of a subject who is a candidate for anti-MICA therapy. MICA overexpression can be naturally occurring or can be induced with DNA damaging agents.

In another embodiment, a method of monitoring a course of treatment in a subject receiving anti-MICA therapy is provided. The method can include determining whether the level of sMICA in the subject after treatment includes a reduced level of sMICA as compared to the level of sMICA in a control sample obtained from the subject at an earlier point in time, where the reduced level indicates that the anti-MICA therapy reduced the level of sMICA in the subject. In another aspect, a combination of other measures of MICA-mediated anti-tumor response may also be included in evaluating a course of treatment, including, for example, increases in NKG2D expression on NK cells and CD8+ T cells, increased NK and CD8+ cytotoxicity, and cross-presentation of MICA expressing tumor cells.

In another embodiment, a method of monitoring an individual at risk for the progression of a pre-malignant plasma cell disorder is provided. The method includes: providing a biological sample from the individual; and determining the level of MICA or anti-MICA antibodies in the biological sample; and comparing the measured level of MICA or anti-MICA antibodies with the level of MICA or anti-MICA antibodies in a control sample, wherein the presence of an altered level of MICA or anti-MICA antibodies in the individual's biological sample compared to the control sample indicates that the individual is at risk for progression of the pre-malignant plasma cell disorder. The biological sample can include blood, serum, plasma cells, or peripheral blood mononuclear cells; the MICA can include soluble MICA or cell-associated MICA. IN another embodiment, the method includes: providing a biological sample from the individual; and determining the level of ERp5 or anti-ERp5 antibodies in the biological sample; and comparing the measured level of ERp5 or anti-ERp5 antibodies with the level of ERp5 or anti-ERp5 antibodies in a control sample, wherein the presence of an altered level of ERp5 or anti-ERp5 antibodies in the individual's biological sample compared to the control sample indicates that the individual is at risk for progression of the pre-malignant plasma cell disorder.

In another embodiment, a method of treating a cancer, or a symptom of cancer or cancer progression in a subject, the method comprising administering to the subject an effective amount of an Erp5-modulating composition. The ERp5 modulating composition can include an anti-ERp5 antibody. In another embodiment, a method of treating a cancer, or a symptom of cancer or cancer progression in a subject, the method comprising administering to the subject an effective amount of a PDI-modulating composition. The PDI-modulating composition can include an anti-PDI antibody.

In another embodiment, a composition including a MICA modulating composition in a pharmaceutically acceptable carrier is provided. The MICA modulating composition can include an anti-MICA antibody, an anti-protein disulfide isomerase (PDI) antibody or a combination thereof. The PDI can be ERp5. In another aspect, the MICA modulating composition can include an agent that modulates MICA shedding. The agent that modulates MICA shedding can include a protein disulfide isomerase (PDI) inhibitor; the PDI can be ERp5. The antibody can be a monoclonal antibody, a polyclonal antibody, an Fab fragment, a chimeric antibody, a humanized antibody, or a single chain antibody. Regardless of the precise molecular form of the anti-MICA antibody, the anti-MICA antibody is a pharmaceutically pure antibody.

In another aspect, the composition can further include one or more tumor cell antigens that elicit an immune response against a tumor. The tumor cell antigens can include autologous tumor cells. The autologous tumor cells can express GM-CSF. In another aspect, the composition can further include an anti-CTLA-4 antibody and one or more tumor cell antigens that elicit an immune response against a tumor. The tumor cell antigens can include autologous tumor cells. The autologous tumor cells can express GM-CSF. In another embodiment, the composition can further include a DNA damaging cancer chemotherapeutic, a proteosome inhibitor or a combination thereof. The proteosome inhibitor can be Bortezomib.

In another embodiment, ERp5 modulating composition in a pharmaceutically acceptable carrier is provided. The ERp5 modulating composition can include an anti-ERp5 antibody. The antibody can be a monoclonal antibody, a polyclonal antibody, an Fab fragment, a chimeric antibody, a humanized antibody, or a single chain antibody. Regardless of the precise molecular form of the anti-ERp5 antibody, the anti-ERp5 antibody is a pharmaceutically pure antibody.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B depict the results of an analysis demonstrating that CTLA-4 blockade following autologous tumor vaccination elicited a potent humoral reaction to MICA that was temporally associated with a reduction in sMICA.

FIGS. 2A and 2B depict the results of an experiment demonstrating that therapy-induced anti-MICA antibodies antagonized sMICA suppression of innate immune responses.

FIGS. 3A and 3B depict the results of an experiment demonstrating that immunotherapy restored protective anti-tumor innate responses in MEL15.

FIGS. 4A, 4B and 4C depict the results of an experiment demonstrating that therapy-induced anti-MICA antibodies antagonized sMICA suppression of adaptive immune responses and enhanced MICA-dependent cross-presentation.

FIGS. 5A, 5B and 5C depict the results of an experiment demonstrating that immunotherapy restored protective anti-tumor innate responses and enhanced cross-presentation in MEL15.

FIGS. 6A and 6B depict the results of an experiment demonstrating that immunotherapy-induced anti-MICA antibodies mediate complement-dependent lysis.

FIGS. 7A, 7B, 7C, 7D and 7E depict the results of an experiment demonstrating that therapy-induced anti-MICA antibodies do not block NK cell lysis of K562 cells.

FIGS. 8A, 8B, 8C, 8D, 8E and 8F depict the results of an experiment demonstrating that vaccination with irradiated, autologous GM-CSF secreting tumor cells alone elicited a potent humoral reaction to MICA that was temporally associated with a reduction in sMICA in some patients.

FIGS. 9A, 9B, and 9C depict the results of an experiment demonstrating that vaccine-induced anti-MICA antibodies antagonized sMICA suppression of innate immune responses.

FIGS. 10A, 10B, and 10C depict the results of an experiment demonstrating that vaccine-induced anti-MICA antibodies antagonized sMICA suppression of adaptive immune responses and enhanced MICA-dependent cross-presentation.

FIG. 11 depicts the results of an experiment demonstrating that the DNA damage response is activated and MICA and ERp5 expression are increased during MGUS/MM progression.

FIGS. 12A, 12B, and 12C depict the results of an experiment analyzing MICA and ERp5 plasma cell surface expression and MICA shedding during the progression of MM.

FIGS. 13A, 13B, and 13C depict the results of an experiment analyzing NKG2D expression and cytotoxic lymphocyte function during the progression of MM.

FIGS. 14A, 14B, 14C and 14D depict the results of an experiment demonstrating that MGUS patients developed anti-MICA antibodies with functional activity.

FIG. 15 depicts the results of an experiment demonstrating that MM, but not MGUS or donor sera inhibited NKG2D dependent NK cell cytotoxicity.

FIGS. 16A, 16B, 16C and 16D depict the results of an experiment demonstrating that Bortezomib activated the DNA damage response and increases MICA expression in some MM cells.

FIG. 17 depicts the results of an experiment demonstrating that Bortezomib induced ATM phosphorylation in U226 cells, but triggers the degradation of ATM in MM1S cells.

FIG. 18 depicts the results of an experiment demonstrating that vaccination with irradiated, GM-CSF secreting RENCA cells stimulated potent anti-tumor humoral immunity.

FIG. 19 depicts the results of an experiment demonstrating that RENCA vaccine targets showed enhanced expression in tumor cells compared to normal tissues.

FIG. 20 depicts the results of an experiment demonstrating that anti-human PDI antibodies were associated with the induction of a clinical response in a patient with acute myeloid leukemia.

FIG. 21 depicts the results of an experiment demonstrating that humoral responses to ERp5 were associated with immune-mediated tumor destruction in diverse solid and hematologic malignancies.

### DETAILED DESCRIPTION

The activation of NKG2D, a cell-surface receptor involved in immune surveillance, on innate and adaptive cytotoxic lymphocytes contributes to immune-mediated tumor destruction. Tumor cell shedding of NKG2D ligands, such as MHC class I chain-related protein A (MICA), results in immune suppression through down-regulation of NKG2D surface expression. The experiments described in the examples indicate that some patients who responded to antibody-blockade of cytotoxic T lymphocyte-associated antigen 4 (CTLA-4) or vaccination with lethally irradiated, autologous tumor cells engineered to secrete granulocyte-macrophage colony stimulating factor (GM-CSF), generated high titer antibodies against MICA. These humoral reactions were associated with a reduction of circulating soluble MICA (sMICA) and an augmentation of NK cell and CD8+ T lymphocyte cytotoxicity. The immunotherapy-induced anti-MICA antibodies efficiently opsonize cancer cells for dendritic cell cross-presentation, which is correlated with a diversification of tumor antigen recognition. The anti-MICA antibodies also accomplish tumor cell lysis through complement fixation. The experiments described in the examples further indicate that ERp5, a protein disulfide isomerase (PDI) involved in MICA shedding, also evoked potent humoral reactions in diverse solid and hematologic malignancy patients who responded to GM-CSF secreting tumor cell vaccines or antibody blockade of cytotoxic T lymphocyte-associated antigen 4 (CTLA-4). In addition, high titer antibodies to another human PDI were similarly induced in an acute myeloid leukemia patient who achieved a complete response following vaccination with irradiated, autologous GM-CSF secreting tumor cells in the setting of non-myeloablative allogeneic bone marrow transplantation.

Disclosed herein are materials and methods relating to the production and use of MICA-modulating (e.g., MICA-inhibiting) compositions for the treatment, inhibition, and management of diseases and disorders associated with MICA overexpression as well as the treatment, inhibition, and management of symptoms of such diseases and disorders. In some embodiments, the MICA-modulating composition includes one or more anti-MICA antibodies. In some embodiments, the MICA-modulating composition includes one or more anti-PDI antibodies. In some embodiments, the MICA-modulating composition includes one or more anti-PDI antibodies and one or more anti-MICA antibodies. The anti-PDI antibodies can be anti-ERp5 antibodies. In some embodiments, the anti-MICA antibodies can be administered along with, after or prior to other antibodies, e.g., anti-CTLA-4 antibodies, anti-PDI (e.g., anti-ERp5 antibodies) and/or other immunotherapies, e.g., vaccination with autologous tumor cells. Also provided are materials and methods relating to the production and use of ERp5-modulating (e.g., ERp5-inhibiting) compositions for the treatment, inhibition, and management of diseases and disorders associated with ERp5 overexpression as well as the treatment, inhibition, and management of symptoms of such diseases and disorders. In some embodiments, the ERp5-modulating composition includes one or more anti-ERp5 antibodies. Such methods may be used to enhance both clinical immunotherapy and conventional cancer therapies, for example, treatments that involve DNA-damaging agents.

### MICA-Modulating Compositions

Provided herein are MICA-modulating compositions. As used herein, the term “modulating” refers to an increase or decrease in the level of MICA relative to the levels of MICA in a biological sample that has not been exposed to the MICA-modulator. MICA is an MHC class I-related polypeptide detected in some normal gastrointestinal epithelial cells and thymocytes. Preferred modulators are inhibitors of one or more activities of MICA. The amino acid sequence of a representative human MICA polypeptide (GenBank accession number NP_{—}000238 (GI:4557751)) is shown in Example 6. Other representative forms of MICA have an amino acid sequence that has 1, 2, 3, 4, 5, 10 or more amino acid changes compared to the amino acid sequence of GenBank Accession No. NP_{—}000238 (GI:4557751). Other amino acid sequences that have been identified for MICA include for example, without limitation, GenBank accession number L14848 (GI:508491) and GenBank accession number AAO45822 (GI:28630987).

Double stranded DNA breaks trigger high-level expression of MICA in a broad range of human cancers, including melanoma, lung, breast, kidney, ovarian, prostate, gastric, pancreatic and colon carcinomas as well as plasma cell cancer, leukemias and lymphomas. MICA is typically localized on the cell surface or in the cytoplasm. MICA is also shed by tumor cells, i.e., released from the cell surface into the surrounding medium, and sera from cancer patients typically contain elevated levels of the soluble form (sMICA). Shed MICA is thought to impair host defense by inducing the internalization of NKD2G molecules on lymphocytes. Shedding generally involves the cleavage and release of a soluble ectodomain from membrane bound pro-proteins; MICA shedding is promoted by a protein disulfide isomerase (PDI), ERp5. PDIs are localized in the endoplasmic reticulum or cell surface where they catalyse reactions that are involved in native disulphide bond formation. The surface localization of ERp5 renders the α3 domain of MICA susceptible to proteolysis; the release of soluble ligand in turn provokes the down-regulation of NKG2D.

The amino acid sequence of a representative human PDI polypeptide (GenBank accession number (Genbank accession number EAW89696; gi:119610102) is shown in Example 15. Other representative forms of human PDI have an amino acid sequence that has 1, 2, 3, 4, 5, 10 or more amino acid changes compared to the amino acid sequence of GenBank Accession No. (Genbank accession number EAW89696; gi:119610102). The amino acid sequence of a representative human ERp5 (also known as PDIA6) polypeptide (GenBank accession AAH01312; gi:1265493) is shown in Example 17. Other representative forms of human ERp5 have an amino acid sequence that has 1, 2, 3, 4, 5, 10 or more amino acid changes compared to the amino acid sequence of GenBank Accession No. AAH01312; gi:1265493.

### Antibodies

A MICA-modulating composition can include anti-MICA antibody or and anti-PDI antibody, for example, an anti-ERp5 antibody. As used herein, useful antibodies can include: monoclonal and polyclonal antibodies, single chain antibodies, chimeric antibodies, bifunctional/bispecific antibodies, humanized antibodies, human antibodies, and complementary determining region (CDR)-grafted antibodies, that are specific for the target protein or fragments thereof, and also include antibody fragments, including Fab, Fab′, F(ab′)2, scFv, Fv, camelbodies, or microantibodies.

Monoclonal antibodies are homogeneous antibodies of identical antigenic specificity produced by a single clone of antibody-producing cells. Polyclonal antibodies generally can recognize different epitopes on the same antigen and that are produced by more than one clone of antibody producing cells, Each monoclonal antibody is directed against a single determinant on the antigen. In addition to their specificity, the monoclonal antibodies are advantageous in that they may be synthesized uncontaminated by other antibodies. The modifier, monoclonal, indicates the character of the antibody as being obtained from a substantially homogeneous population of antibodies, and is not to be construed as requiring production of the antibody by any particular method. For example, the monoclonal antibodies may be made by the hybridoma method first described by Kohler et al., Nature, 256:495 (1975), or may be made by recombinant DNA methods (see, e.g., U.S. Pat. No. 4,816,567). The monoclonal antibodies may also be isolated from phage antibody libraries using the techniques described in Clackson et al., Nature, 352:624-628 (1991) and Marks et al., J. Mol. Biol., 222:581-597 (1991), for example.

The monoclonal antibodies herein can include chimeric antibodies, i.e., antibodies that typically have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest include primatized antibodies comprising variable domain antigen-binding sequences derived from a non-human primate (e.g. apes, Old World monkeys, New World monkeys, prosimians) and human constant region sequences.

Antibody fragments generally include a portion of an intact antibody. In some embodiments, the portion of an intact antibody can be the antigen-binding or variable region of the corresponding intact antibody. Examples of antibody fragments include Fab, Fab', F(ab')₂, and Fv fragments; diabodies; linear antibodies (Zapata et al., Protein Eng. 8(10): 1057-1062 [1995]); single-chain antibody molecules; and multispecific antibodies formed from antibody fragment(s).

An intact antibody is one that comprises an antigen-binding variable region as well as a light chain constant domain (CO and heavy chain constant domains, C_{H1}, C_{H2} and C_{H3}. The constant domains may be native sequence constant domains (e.g. human native sequence constant domains) or amino acid sequence variants thereof. In some embodiments the intact antibody has one or more effector functions.

A wide variety of antibody/immunoglobulin frameworks or scaffolds can be employed so long as the resulting polypeptide includes at least one binding region that is specific for the target protein. Such frameworks or scaffolds include the five main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

One can generate non-immunoglobulin based antibodies using non-immunoglobulin scaffolds onto which CDRs of the anti-MICA antibody or the anti-PDI antibody, for example, an anti-ERp5 antibody, can be grafted. Any non-immunoglobulin framework and scaffold know to those in the art may be used, as long as the framework or scaffold includes a binding region specific for the target. Examples of non-immunoglobulin frameworks or scaffolds include, but are not limited to, Adnectins (fibronectin) (Compound Therapeutics, Inc., Waltham, Mass.), ankyrin (Molecular Partners AG, Zurich, Switzerland), domain antibodies (Domantis, Ltd (Cambridge, Mass.) and Ablynx nv (Zwijnaarde, Belgium)), lipocalin (Anticalin) (Pieris Proteolab AG, Freising, Germany), small modular immuno-pharmaceuticals (Trubion Pharmaceuticals Inc., Seattle, Wash.), maxybodies (Avidia, Inc. (Mountain View, Calif.)), Protein A (Affibody AG, Sweden) and affilin (gamma-crystallin or ubiquitin) (Scil Proteins GmbH, Halle, Germany).

The term polypeptide as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

The anti-MICA antibody or the anti-PDI antibody, for example, the anti-ERp5 antibody, can be a monoclonal antibody, a polyclonal antibody, a chimeric antibody, a human antibody, a humanized antibody, a single-chain antibody, or an Fab fragment. In some embodiments the antibody has a binding affinity less than about 1×10⁵ Ka for a polypeptide other than MICA or PDI. In some embodiments, the anti-MICA antibody or the anti-PDI antibody, for example, the anti-ERp5 antibody is a monoclonal antibody which binds to MICA, PDI, or ERp5, respectively with an affinity of at least 1×10⁸ Ka.

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, in some embodiments in an adjuvant such as Freund's complete adjuvant, and injecting the mixture or emulsion parenterally. Any method of immunization known in the art may be used to obtain the monoclonal antibodies. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial or limiting dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

In some embodiments the anti-MICA antibody or the anti-PDI antibody, for example, the anti-ERp5 antibody, is a humanized antibody. Human antibodies can be produced using techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boemer et al., J. Immunol., 147(1):86 95 (1991)).

Humanized antibodies may be engineered by a variety of methods including, for example: (1) grafting the non-human complementarity determining regions (CDRs) onto a human framework and constant region (a process referred to in the art as humanizing), or, alternatively, (2) transplanting the entire non-human variable domains, but providing them with a human-like surface by replacement of surface residues (a process referred to in the art as veneering). Humanized antibodies can include both humanized and veneered antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); Jones et al., Nature 321:522-525 (1986); Morrison et al., Proc. Natl. Acad. Sci, U.S.A., 81:6851-6855 (1984); Morrison and 01, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

In addition to chimeric and humanized antibodies, fully human antibodies can be derived from transgenic mice having human immunoglobulin genes (see, e.g., U.S. Pat. Nos. 6,075,181, 6,091,001, and 6,114,598, all of which are incorporated herein by reference), or from phage display libraries of human immunoglobulin genes (see, e.g. McCafferty et al., Nature, 348:552-554 (1990). Clackson et al., Nature, 352:624-628 (1991), and Marks et al., J. Mol. Biol., 222:581-597 (1991)). In some embodiments, antibodies may be produced and identified by scFv-phage display libraries. Antibody phage display technology is available from commercial sources such as from Morphosys.

As an alternative to the use of hybridomas for expression, antibodies can be produced in a cell line such as a CHO or myeloma cell line, as disclosed in U.S. Pat. Nos. 5,545,403; 5,545,405; and 5,998,144; each incorporated herein by reference. Briefly the cell line is transfected with vectors capable of expressing a light chain and a heavy chain, respectively. By transfecting the two proteins on separate vectors, chimeric antibodies can be produced. Immunol. 147:8; Banchereau et al. (1991) Clin. Immunol. Spectrum 3:8; and Banchereau et al. (1991) Science 251:70; all of which are herein incorporated by reference.

A complementarity determining region of an antibody typically includes amino acid sequences that together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). A constant region of an antibody typically includes the portion of the antibody molecule that confers effector functions, including for example, the portion that binds to the Fc receptor on dendritic cells. In some embodiments, mouse constant regions can be substituted by human constant regions. For example, the constant regions of humanized antibodies are derived from human immunoglobulins. The heavy chain constant region can be selected from any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which are incorporated herein by reference.

Human antibodies can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Antibodies can also be produced using human engineering techniques as discussed in U.S. Pat. No. 5,766,886, which is incorporated herein by reference.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of for example, a transgenic mouse as described in WO 96/33735. The monoclonal antibodies can be tested for the ability to inhibit or neutralize the biological activity or physiological effect of the corresponding protein.

Fragments of antibodies are suitable for use in the methods provided so long as they retain the desired affinity and specificity of the full-length antibody. Thus, a fragment of an anti-MICA antibody or the anti-PDI antibody will retain an ability to bind to MICA or PDI, respectively, in the Fv portion and the ability to bind the Fc receptor on dendritic cells in the FC portion. Such fragments are characterized by properties similar to the corresponding full-length anti-MICA antibody or the anti-PDI antibody, that is, the fragments will specifically bind a human MICA antigen or the PDI antigen, respectively, expressed on the surface of a human cell or the corresponding sMICA antigen that has been shed into the media.

Also provided are antibodies that are SMIPs or binding domain immunoglobulin fusion proteins specific for target protein. These constructs are single-chain polypeptides comprising antigen binding domains fused to immunoglobulin domains necessary to carry out antibody effector functions. See e.g., WO03/041600, U.S. Patent publication 20030133939 and US Patent Publication 20030118592.

Any form of the MICA or the PDI polypeptide can be used to generate anti-MICA or anti-PDI antibodies, respectively, including the full length polypeptide or epitope-bearing fragments thereof. Highly suitable anti-MICA or anti PDI antibodies are those of sufficient affinity and specificity to recognize and bind to MICA and sMICA, or PDI, respectively, in vivo. As used herein, the term epitope refers to an antigenic determinant of a polypeptide. In some embodiments an epitope may comprise 3 or more amino acids in a spatial conformation which is unique to the epitope. In some embodiments epitopes are linear or conformational epitopes. Generally an epitope consists of at least 4, at least 6, at least 8, at least 10, and at least 12 such amino acids, and more usually, consists of at least 8-10 such amino acids. Methods of determining the spatial conformation of amino acids are known in the art, and include, for example, x-ray crystallography and 2-dimensional nuclear magnetic resonance.

In some embodiments, the antibodies specifically bind to one or more epitopes in an extracellular domain of MICA or PDI. Suitable antibodies can recognize linear or conformational epitopes, or combinations thereof. It is to be understood that these peptides may not necessarily precisely map to one epitope, but may also contain an MICA or PDI sequence, respectively, that is not immunogenic.

Methods of predicting other potential epitopes to which an antibody can bind are well-known to those of skill in the art and include without limitation, Kyte-Doolittle Analysis (Kyte, J. and Dolittle, R. F., J. Mol. Biol. (1982) 157:105-132), Hopp and Woods Analysis (Hopp, T. P. and Woods, K R., Proc. Natl. Acad. Sci. USA (1981) 78:3824-3828; Hopp, T. J. and Woods, K. R., Mol. Immunol. (1983) 20:483-489; Hopp, T. J., J. Immunol. Methods (1986) 88:1-18.), Jameson-Wolf Analysis (Jameson, B. A. and Wolf, H., Comput. Appl. Biosci. (1988) 4:181-186.), and Emini Analysis (Emini, E. A., Schlief, W. A., Colonno, R. I. and Wimmer, E., Virology (1985) 140:13-20.). In some embodiments, potential epitopes are identified by determining theoretical extracellular domains. Analysis algorithms such as TMpred (see K. Hofmann & W. Stoffel (1993) TMbase—A database of membrane spanning proteins segments Biol. Chem. Hoppe-Seyler 374,166) or TMHMM (A. Krogh, B. Larsson, G. von Heijne, and E. L. L. Sonnhammer. Predicting transmembrane protein topology with a hidden Markov model: Application to complete genomes. Journal of Molecular Biology, 305(3):567-580, January 2001) can be used to make such predictions. Other algorithms, such as SignalP 3.0 (Bednsten et al, (2004) J Mol. Biol. 2004 Jul. 16; 340(4):783-95) can be used to predict the presence of signal peptides and to predict where those peptides would be cleaved from the full-length protein. The portions of the proteins on the outside of the cell can serve as targets for antibody interaction.

Specifically binding antibodies are can be antibodies that 1) exhibit a threshold level of binding activity; and/or 2) do not significantly cross-react with known related polypeptide molecules. The binding affinity of an antibody can be readily determined by one of ordinary skill in the art, for example, by Scatchard analysis (Scatchard, Ann. NY Acad. Sci. 51: 660-672, 1949). In some embodiments the antibodies can bind to their target epitopes or mimetic decoys at least 1.5-fold, 2-fold, 5-fold 10-fold, 100-fold, 10³-fold, 10⁴-fold, 10⁵-fold, 10⁶-fold or greater for the target cancer-associated polypeptide than to other proteins predicted to have some homology to MICA.

In some embodiments the antibodies bind with high affinity of 10⁻⁴ M or less, 10⁻⁷ M or less, 10⁻⁹ M or less or with subnanomolar affinity (0.9, 0.8, 0.7, 0.6, 0.5, 0.4, 0.3, 0.2, 0.1 nM or even less). In some embodiments the binding affinity of the antibodies for MICA is at least 1×10⁶ Ka. In some embodiments the binding affinity of the antibodies for MICA is at least 5×10⁶ Ka, at least 1×10⁷ Ka, at least 2×10⁷ Ka, at least 1×10⁸ Ka, or greater. Antibodies may also be described or specified in terms of their binding affinity to a MICA polypeptide. In some embodiments binding affinities include those with a K_{d} less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹¹ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the antibodies do not bind to known related polypeptide molecules; for example, they bind MICA polypeptide or PDI polypeptide, respectively, but not known related polypeptides using a standard immunoblot analysis (Ausubel et al., Current Protocols in Molecular Biology, 1994).

In some embodiments, antibodies may be screened against known related polypeptides to isolate an antibody population that specifically binds to MICA or PDI polypeptides, respectively. For example, antibodies specific to human MICA polypeptides will flow through a column comprising MICA-related proteins (with the exception of MICA) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current Protocols in Immunology, Cooligan et al. (eds.), National Institutes of Health, John Wiley and Sons, Inc., 1995). Screening and isolation of specific antibodies is well known in the art (see, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984). Representative examples of such assays include: concurrent immunoelectrophoresis, radioimmunoassay (MA), radioimmunoprecipitation, enzyme-linked immunosorbent assay (ELISA), dot blot or Western blot assay, inhibition or competition assay, and sandwich assay.

Antibodies can be purified by chromatographic methods known to those of skill in the art, including ion exchange and gel filtration chromatography (for example, Caine et al., Protein Expr. Purif (1996) 8(2):159-166). Alternatively or in addition, antibodies can be purchased from commercial sources, for example, Invitrogen (Carlsbad, Calif.); MP Biomedicals (Solon, Ohio); Nventa Biopharmaceuticals (San Diego, Calif.) (formerly Stressgen); Serologicals Corp. (Norcross, Ga.).

The MICA-modulator can include a monoclonal antibody that recognizes a single MICA epitope or can be any combination of monoclonal or polyclonal antibodies recognizing one of more different MICA epitopes. Thus the MICA-modulator can include antibodies recognize 2, 3, 4, 5, 6, 7, 8, 10, 20 or more different MICA epitopes. The MICA-modulator can include a monoclonal antibody that recognizes a single PDI epitope or can be any combination of monoclonal or polyclonal antibodies recognizing one of more different PDI epitopes. Thus the PDI-modulator can include antibodies recognize 2, 3, 4, 5, 6, 7, 8, 10, 20 or more different PDI epitopes.

In some embodiments, antibodies may act as MICA antagonists. For example, in some embodiments the antibodies can disrupt the receptor/ligand interactions with MICA either partially or fully. In some embodiments, antibodies are provided that modulate ligand activity or receptor activity by at least 95%, at least 90%, at least 85%, at least 80%, at least 75%, at least 70%, at least 60%, or at least 50% compared to the activity in the absence of the antibody. In some embodiments, antibodies may act as PDI antagonists, for example, as ERp5 antagonists. For example, in some embodiments the antibodies can disrupt the receptor/ligand interactions with PDI, for example ERp5, either partially or fully. In some embodiments, antibodies are provided that modulate ligand activity or receptor activity by at least 95%, at least 90%, at least 85%, at least 80%, at least 75%, at least 70%, at least 60%, or at least 50% compared to the activity in the absence of the antibody.

In some embodiments neutralizing antibodies are provided. In some embodiments the neutralizing antibodies act as receptor antagonists, i.e., inhibiting either all or a subset of the biological activities of the ligand-mediated receptor activation. In some embodiments the antibodies may be specified as agonists, antagonists or inverse agonists for biological activities comprising the specific biological activities of the peptides disclosed herein.

In some embodiments, the MICA-modulating composition can include a combination of anti-MICA antibodies, anti-PDI antibodies, for example anti-ERp5 antibodies, and antibodies against cytotoxic lymphocyte antigen-4 (CTLA-4). CTLA-4 is a cytotoxic T-lymphocyte-associated granule serine protease that appears to be involved in T-cell activation. Amino acid sequences of representative human CTLA-4 polypeptides include for example, without limitation, GenBank numbers NM_{—}005214, and NM_{—}001037631. Binding of CTLA-4 to ligands B7-1 (CD80) and B7-2 (CD86) induces cell cycle arrest and diminished cytokine production. Transient blocking CTLA-4 activity with anti-CTLA-4 antibodies (“CTLA-4 blockade”) enhances antigen specific T-cell responses with limited toxicity.

The anti-CTLA-4 antibody can be a monoclonal antibody, a chimeric antibody, a human antibody, a humanized antibody, a single-chain antibody, or an Fab fragment. Anti-CLTA-4 antibodies can be prepared as described above. Any CTLA-4 epitope can be used to generate the antibodies, provided that the resulting antibody binds to CTLA-4 in vivo in such a way that it blocks the binding of the CTLA-4 ligands, B7-1 and B7-2. Blocking antibodies can be identified based on their ability to compete with labeled ligands B7-1 and B7-2 for binding to CTLA-4 using standard screening methods.

In some embodiments, the MICA-modulating composition can include an anti-cancer vaccine. Typically, cancer vaccines are designed to treat cancer by stimulating the immune system to recognize and attack human cancer cells without harming normal cells. A cancer vaccine can include one or more tumor antigens that elicit an immune response against a tumor. For example, a useful method for eliciting an immune response against a tumor in a patient can include immunization with irradiated autologous GM-CSF-secreting tumor cells. In this method, a killed sample of the patient's own tumor cells that have been genetically engineered to express the immuno-stimulating cytokine, GM-CSF, is used to stimulate an immune response against a patient's tumor. Methods for the production of irradiated autologous GM-CSF-secreting tumor cells and the use of irradiated autologous GM-CSF-secreting tumor cells to stimulate an immune response against cancer have been described in Soiffer R, Lynch T, Mihm M, Jung K, Rhuda C, Schmollinger J C, Hodi F S, Liebster L, Lam P, Mentzer S, Singer S, Tanabe K K, Cosimi A B, Duda R, Sober A, Bhan A, Daley J, Neuberg D, Parry G, Rokovich J, Richards L, Drayer J, Bems A, Clift S, Cohen L K, Mulligan R C, Dranoff G. “Vaccination with irradiated, autologous melanoma cells engineered to secrete human granulocyte-macrophage colony stimulating factor generates potent anti-tumor immunity in patients with metastatic melanoma”. Proc. Natl. Acad. Sci. USA 1998; 95:13141-13146 and in Soiffer R J, Hodi F S, Haluska F, Jung K, Gillessen S, Singer S, Tanabe K, Duda R, Mentzer S, Jaklitsch M, Bueno R, Clift S, Hardy S, Neuberg D, Mulligan R C, Webb I, Mihm M, Dranoff G. “Vaccination with irradiated, autologous melanoma cells engineered to secrete granulocyte-macrophage colony stimulating factor by adenoviral mediated gene transfer augments anti-tumor immunity in patients with metastatic melanoma.” J. Clin. Oncol. 2003; 21:3343-3350.

### Activity of MICA-Modulating Compositions

Without being limited by any particular theory, it appears that MICA-modulating compositions can function through the two major components of the mammalian immune system: the innate immune system and the adaptive immune system. As used herein, the innate immune system refers to all non-specific host defense mechanisms. The innate immune system includes both physical barriers, for example, skin, gastric acid, mucus or tears, as well as cells and active mechanisms such as Natural Killer (NK) cells, phagocytes and the complement system. Natural killer cells or (NK) cells are a major component of the innate immune system. NK cells are cytotoxic, attacking cells that have been infected by microbes as well as some kinds of tumor cells. The cytotoxic activity of NK cells is mediated through cell surface receptors that recognize MHC class I alleles. Receptor types include CD94:NKG2, Ly49, KIR (Killer cell Immunoglobulin-like Receptors) and ILT or LIR (leucocyte inhibitory receptors). MICA is a ligand for one receptor subtype, NKG2D. Phagocytic cells include neutrophils, monocytes, macrophages, basophils and eosinophils. The complement system is a biochemical cascade of the immune system that helps clear pathogens from an organism. It is derived from many small plasma proteins working together to form the primary end result of cytolysis by disrupting the target cell's plasma membrane. The proteins are sythesized in the liver, mainly by hepatocytes.

The adaptive immune system, as used herein, refers to specific antibody production by B lymphocytes and antigen-specific activity by T lymphocytes. The humoral response, mediated by B lymphocytes, defends primarily against extracellular pathogens through the production of circulating antibodies that mark foreign cells and molecules for destruction by other specialized cells and proteins. The cellular response, mediated by T lymphocytes, defends predominantly against intracellular pathogens and cancers by directly binding to and destroying the infected or cancerous cells. Both responses depend upon specialized cells that internalize through endocytosis, pinocytosis or phagocytosis, and process immunogens; fragments of the immunogens are then presented to T lymphocytes, which in turn, help to trigger B-lymphoctye responses against the immunogens.

In some embodiments, a MICA modulating composition can be an opsonizing agent. Opsonization is the process where cells or particles become coated with molecules which allow them to bind to receptors on other cell types, e.g., dendritic cells or phagocytes to promote their uptake. For antigen presenting cells such as dendritic cells and macrophages, opsonization promotes efficient antigen processing and presentation. Antibodies (especially IgG) can opsonize and are therefore referred to as “opsonins”. Opsonizing agents that are capable of specifically binding both the target (i.e., MICA) and particular receptors on antigen presenting cells (e.g., Fc receptors) that lead to internalization and subsequent antigen processing/presentation are particularly useful.

MICA-bearing tumor cells can also become opsonized, i.e., coated with anti-MICA antibodies. For example, IgG antibodies bind to MICA on the tumor cell surface through the Fab region, leaving the Fc region exposed. Dendritic cells have Fc gamma receptors and therefore they can bind to and internalize the MICA tumor antigen, and then present the MICA antigens to CD8+ T cells. As described herein, the term “cross-presentation” (also known as cross-priming) denotes the ability of certain antigen-presenting cells to take up, process and present extracellular antigens with MHC class I molecules to CD8+ T cells (cytotoxic T cells). Opsonization will similarly result in the generation of MHC class II restricted CD4+ T cell responses.

Thus, the MICA-modulating compositions can have multiple therapeutic functions, including for example, antigen-binding, complement-dependent cellular cytotoxicity (CDC) as well as antibody-dependent cellular cytotoxicity (ADCC), a lytic attack on antibody-targeted cells, and potentially, the induction of apoptosis.

### Other NKG2D Ligands

In some cases it may be desirable to administer a composition that modulates the activity of an NKG2D ligand other than MICA. For example, MICB (GenBank® Accession No. NM_{—}00593; GI: 26787987), ULBP-1 (GenBank® Accession No. NP_{—}079494; GI:13376826), ULBP-2 (GenBank® Accession No. NP_{—}079493; GI:133768264), ULBP-3 (GenBank® Accession No. NP_{—}078794; GI:13337565600) and ULBP-4 (also known as LETAL or RAFT-IE; GenBank® Accession No. NM_{—}139165; GI:21040248) are NKG2D ligands. Antibodies selective for one or more of these ligands can be administered to a patient in conjunction with an antibody selective for MICA. In addition, an antibody selective for one or more of these ligands can be administered in place of an antibody selective for MICA under conditions when tumor cells express these ligands and/or shed them into the blood.

### Methods of Treating/Preventing Cancer

Provided herein are methods for treating and/or preventing cancer or symptoms of cancer in a subject comprising administering to the subject a therapeutically effective amount of a MICA-modulating composition. The MICA modulating composition can include one or more anti-MICA antibodies and one or more anti-PDI, for example, anti-Erp5, antibodies. The methods disclosed herein are generally useful for generating immune responses and as prophylactic vaccines or immune response-stimulating therapeutics. As used herein, “prophylaxis” can mean complete prevention of the symptoms of a disease, a delay in onset of the symptoms of a disease, or a lessening in the severity of subsequently developed disease symptoms. As used herein, “therapy” can mean a complete abolishment of the symptoms of a disease or a decrease in the severity of the symptoms of the disease. In some embodiments the cancer is a cancer associated with overexpression of MICA. In some embodiments, the cancer is melanoma, lung, breast, kidney, ovarian, prostate, pancreatic, gastric, and colon carcinoma, lymphoma or leukemia. In some embodiments, the cancer is melanoma. In some embodiments, the cancer is a plasma cell malignancy, for example, multiple myeloma (MM) or pre-malignant condition of plasma cells. In some embodiments the subject has been diagnosed as having a cancer or as being predisposed to cancer.

The materials and methods disclosed herein are useful therapeutics for the treatment of pre-malignant disorders that carry with them a risk of progression to malignancy. Examples of such disorders include, without limitation, dysplasia, hyperplasia, and plasma cell disorders such as monoclonal gammopathy of undetermined significance (MGUS) and smoldering multiple myeloma (SMM). MGUS and SMM are asymptomatic, pre-malignant disorders characterized by monoclonal plasma cell proliferation in the bone marrow and absence of end-organ damage such as osteolytic bone lesions, anemia, or renal failure. The risk of progression to MM is about 1% per year for MGUS and 10-20% per year for SMM. The mechanisms underlying the progression from MGUS to MM are incompletely understood, but include the suppression of innate and adaptive anti-tumor immunity. Patients with MGUS or SMM require indefinite follow-up given their life-long risk of progression to MM or related malignancy.

Symptoms of cancer are well-known to those of skill in the art and include, without limitation, unusual mole features, a change in the appearance of a mole, including asymmetry, border, color and/or diameter, a newly pigmented skin area, an abnormal mole, darkened area under nail, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreatic metastases, difficulty swallowing, and the like.

The methods disclosed herein can be applied to a wide range of species, e.g., humans, non-human primates (e.g., monkeys), horses, cattle, pigs, sheep, deer, elk, goats, dogs, cats, mustelids, rabbits, guinea pigs, hamsters, rats, and mice.

The compositions can be administered directly to a mammal. Generally, the antibodies can be suspended in a pharmaceutically-acceptable carrier (e.g., physiological saline). A composition can be made by combining any of the MICA-modulating compositions provided herein with a pharmaceutically acceptable carrier. Such carriers can include, without limitation, sterile aqueous or non-aqueous solutions, suspensions, and emulsions. Examples of non-aqueous solvents include mineral oil, propylene glycol, polyethylene glycol, vegetable oils, and injectable organic esters, for example. Aqueous carriers include, without limitation, water, alcohol, saline, and buffered solutions. Preservatives, flavorings, and other additives such as, for example, antimicrobials, anti-oxidants, chelating agents, inert gases, and the like also may be present. It will be appreciated that any material described herein that is to be administered to a mammal can contain one or more pharmaceutically acceptable carriers.

Any composition described herein can be administered to any part of the host's body. A composition can be delivered to, without limitation, the joints, nasal mucosa, blood, lungs, intestines, muscle tissues, skin, or peritoneal cavity of a mammal. In addition, a composition can be administered by intravenous, intraperitoneal, intramuscular, subcutaneous, intramuscular, intrarectal, intravaginal, intrathecal, intratracheal, intradermal, or transdermal injection, by oral or nasal administration, by inhalation, or by gradual perfusion over time. In a further example, an aerosol preparation of a composition can be given to a host by inhalation.

The dosage required depends on the route of administration, the nature of the formulation, the nature of the patient's illness, the subject's size, weight, surface area, age, and sex, other drugs being administered, and the judgment of the attending physician. Suitable dosages are in the range of 0.01-1,000 μg/kg. Wide variations in the needed dosage are to be expected in view of the variety of MICA-modulating compositions available and the differing efficiencies of various routes of administration. Variations in these dosage levels can be adjusted using standard empirical routines for optimization as is well understood in the art. Administrations can be single or multiple (e.g., 2- or 3-, 4-, 6-, 8-, 10-, 20-, 50-, 100-, 150-, or more fold). Encapsulation of the composition in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, MICA-modulating compositions can be administered once a month for three months or once a year for a period of ten years. It is also noted that the frequency of treatment can be variable. For example, MICA-modulating compositions can be administered once (or twice, three times, etc.) daily, weekly, monthly, or yearly. MICA modulating compositions can be administered together, i.e., at the same point in time or sequentially. For example, a patient can receive an autologous tumor cell vaccine followed by an anti-CTL4 antibody, followed by an anti-MICA antibody, separated by intervals of hours, days, months or years.

Alternatively or in addition the compositions can be administered along with an adjuvant. An “adjuvant” is an immunological compound that can enhance an immune response against a particular antigen such as a polypeptide. Examples of adjuvants include alum and other aluminum-based compounds (e.g., Al₂O₃). Aluminum-based compounds can be obtained from various commercial suppliers. Other adjuvants include immuno-stimulating complexes (ISCOMs) that can contain such components as cholesterol and saponins; one or more additional immunostimulatory components, including, without limitation, muramyldipeptide (e.g., N-acetylmuramyl-L-alanyl-D-isoglutamine; MDP), monophosphoryl-lipid A (MPL), and formyl-methionine containing tripeptides such as N-formyl-Met-Leu-Phe. Such compounds are commercially available from Sigma Chemical Co. (St. Louis, Mo.) and RIBI ImmunoChem Research, Inc. (Hamilton, Mont.), for example. Other adjuvants can include CpG oligodeoxynucleotides (Coley Pharmaceuticals), QS21 (Cambridge Biotech) and MF59 (Chiron). Adjuvants that enhance dendritic cell function can also be used; examples include GM-CSF, Flt3-ligand, and interferons.

The compositions provided herein can contain any ratio of adjuvant to antibody. The adjuvant:antibody ratio can be 50:50 (vol:vol), for example. Alternatively, the adjuvant:antibody ratio can be, without limitation, 90:10, 80:20, 70:30, 64:36, 60:40, 55:45, 40:60, 30:70, 20:80, or 90:10.

An effective amount of any composition provided herein can be administered to a host. The term “effective” as used herein refers to any amount that induces a desired immune response while not inducing significant toxicity in the host. Such an amount can be determined by assessing a host's immune response after administration of a known amount of a particular composition. In addition, the level of toxicity, if any, can be determined by assessing a host's clinical symptoms before and after administering a known amount of a particular composition. It is noted that the effective amount of a particular composition administered to a host can be adjusted according to a desired outcome as well as the host's response and level of toxicity. Significant toxicity can vary for each particular host and depends on multiple factors including, without limitation, the host's disease state, age, and tolerance to pain.

Antibodies can also be administered to a subject via in vivo therapeutic antibody gene transfer as discussed by Fang et al. (2005), Nat. Biotechnol. 23, 584-590. For example recombinant vectors can be generated to deliver a multicistronic expression cassette comprising a peptide that mediates enzyme independent, cotranslational self cleavage of polypeptides placed between MAb heavy and light chain encoding sequences. Expression leads to stoichiometric amounts of both MAb chains. In some embodiments the peptide that mediates enzyme independent, cotranslational self cleavage is the foot-and-mouth-disease derived 2A peptide.

Any method can be used to determine if a particular immune response is induced. For example, antibody responses against MICA or PDI, for example, ERp5, can be determined using an immunological assay (e.g., ELISA or lymphocyte proliferation assay). In such an assay, the wells of a microtiter plate can be coated with MICA or PDI, for example, ERp5, and incubated with serum from a mammal treated with the immune conjugate designed to produce antibodies against the corresponding immunogen in that mammal, and the presence or absence of antibodies against the immunogen can be determined by standard methods know to those in the art. Other methods to monitor induction of an anti-MICA response include for example, without limitation, one of increased NKG2D-dependent cell killing, increased CD8+ T-lymphocyte toxicity, and MICA dependent complement fixation. In addition, levels of sMICA in a patient's serum can be monitored by ELISA. A decrease in the levels of sMICA by 2%, 5%, 10%, 20%, 50%, 80% or more can be indicative of an immune response to MICA and can correlate with a regression in clinical symptoms.

In addition, clinical methods that can assess the degree of a particular disease state can be used to determine if a desired immune response is induced. For example, in a cancer patient, a reduction in tumor burden or a delay in the recurrence or metastasis can indicate a desired immune response in a patient treated with a MICA-modulating composition.

Also provided are methods of inhibiting cancer in a patient. The methods comprise determining if the patient is a candidate for MICA therapy as described herein and administering a therapeutically effective amount of one or more MICA modulators to the patient if the patient is a candidate for MICA therapy. Further provided are methods of inhibiting cancer in a patient diagnosed or suspected of having a cancer. The methods comprise administering a therapeutically effective amount of one or more MICA modulators to the patient. Also provide are methods of modulating one or more symptoms of cancer in a patient comprising administering to said patient a therapeutically effective amount of one or more MICA modulators.

Methods to prophylactically treat a patient who is predisposed to develop cancer, a cancer metastasis or who has had a metastasis and is therefore susceptible to a relapse or recurrence are disclosed. The methods are particularly useful in high-risk individuals who, for example, have a family history of cancer or of metastasizing tumors, or show a genetic predisposition for a cancer metastasis. In some embodiments the tumors are MICA-related tumors. Additionally, the methods are useful to prevent patients from having recurrences of MICA-related tumors who have had MICA-related tumors removed by surgical resection or treated with a conventional cancer treatment. Also provided are methods of inhibiting cancer progression and/or causing cancer regression comprising administering to the patient a therapeutically effective amount of an MICA modulator.

In some embodiments, the patient in need of anti-cancer treatment can be treated with the MICA modulators described herein in conjunction with one or more antibodies directed at targets other than MICA. Suitable targets can include cancer cell surface molecules, e.g., the EGF receptor, VEGF, HER-2, CD20, c-Met, ErbB3, angiopoietins, and gangliosides such as GM2.

In some embodiments, the patient in need of anti-cancer treatment is treated with the MICA modulators described herein in conjunction with chemotherapy and/or radiation therapy. For example, following administration of the MICA modulators, the patient may also be treated with a therapeutically effective amount of anti-cancer radiation. In some embodiments chemotherapeutic treatment is provided in combination with MICA modulators. In some embodiments MICA modulators are administered in combination with chemotherapy and radiation therapy.

Methods of treatment comprise administering single or multiple doses of one or more MICA modulators to the patient. In some embodiments the MICA modulators are administered as injectable pharmaceutical compositions that are sterile, pyrogen free and comprise the MICA modulators in combination with a pharmaceutically acceptable carrier or diluent.

In some embodiments, the therapeutic regimens described herein are used with conventional treatment regimens for cancer including, without limitation, surgery, radiation therapy, hormone ablation and/or chemotherapy. Administration of the MICA modulators described herein may take place prior to, simultaneously with, or after conventional cancer treatment. In some embodiments, two or more different MICA modulators are administered to the patient.

Also provided are methods of monitoring the progression of pre-malignant disorders that have the potential for progression to malignancy, for example, plasma cell disorders such as monoclonal gammopathy of undetermined significance (MGUS) and smoldering multiple myeloma (SMM). More specifically, a patient having a pre-malignant plasma cell disorder can be identified as being at risk for progression of the pre-malignant plasma cell disorder to a malignancy by assessing the levels of MICA or anti-MICA antibodies in the individual. MICA can be either cell-associated MICA, i.e., intracellular or cell-surface MICA, or sMICA. In some embodiments, an individual who does not express or who expresses low levels of cell-associated MICA or anti-MICA antibodies relative to a reference sample can be classified as being at risk for progression to malignancy. In some embodiments, an individual who expresses elevated levels of sMICA relative to a reference sample can be classified as being at risk for progression to malignancy. In some embodiments, a patient having a pre-malignant plasma cell disorder can be identified as being at risk for progression of the pre-malignant plasma cell disorder to a malignancy by assessing the levels of PDI or anti-PDI antibodies in the individual. In some embodiments, a patient having a pre-malignant plasma cell disorder can be identified as being at risk for progression of the pre-malignant plasma cell disorder to a malignancy by assessing the levels of ERp5 or anti-ERp5 antibodies in the individual.

The level of MICA or anti-MICA antibodies can be measured in any biological sample known in the art to comprise MICA or anti-MICA antibodies. Examples of biological samples include, without limitation, whole blood, serum, blood plasma, peripheral blood mononuclear cells (PBMC's) and bone marrow aspirates. Biological samples can be collected from an individual using any standard method known in the art that results in the preservation of MICA or anti-MICA antibodies. Blood samples can be obtained via venous puncture techniques. Serum samples can be prepared from whole blood using standard methods such as centrifuging blood samples that have been allowed to clot. Plasma samples can be obtained by centrifuging blood samples that were treated with an anti-coagulant such as heparin. PBMC's and bone marrow aspirates can be processed by Ficoll-Hypaque density gradient centrifugation. Biological samples can be assayed for MICA or anti-MICA antibodies immediately following collection. Alternatively, or in addition, a biological fluid sample can be stored for later analysis using methods known in the art that preserve MICA or anti-MICA antibodies, e.g., freezing, drying, freeze drying.

After determining the levels of MICA or anti-MICA antibodies in a biological sample, these levels can be compared with those of a control sample. A control sample can be a one or more samples taken from the same individual at a earlier point in time. Alternatively or in addition a control sample can be a standard reference level. Standard reference levels typically represent the average MICA or anti-MICA antibody levels derived from a large population of individuals. The reference population may include individuals of similar age, body size, ethnic background or general health as the individual in question.

In general, an elevated level of MICA or anti-MICA antibodies can be any level of MICA or anti-MICA antibodies that is greater than either the level of MICA or anti-MICA antibodies found in a control sample or the average level of MICA or anti-MICA antibodies found in samples from a population of normal healthy individuals. A reduced level of MICA or anti-MICA antibodies can be any level of MICA or anti-MICA antibodies antigen that is less than either the level of MICA or anti-MICA antibodies found in a control sample or the average level of MICA or anti-MICA antibodies found in samples from a population of normal healthy individuals. Any population size can be used to determine the average level of MICA or anti-MICA antibodies found in samples from a population of normal healthy individuals. For example, a population of 2, 3, 4, 5, 10, 15, 20, 25, 30, 40, 50, 100, 150, 200, 250 or more individuals can be used to determine the average level of MICA or anti-MICA antibodies in samples from a population of normal healthy individuals.

An elevated level of MICA or anti-MICA antibodies can be 1, 2, 3, 4, 5, 10, 20, or more percent higher than that level found in a control sample or the average level of MICA or anti-MICA antibodies found in samples from a population of normal healthy individuals. In some cases, an elevated level of MICA or anti-MICA antibodies can be 1, 2, 3, 4, 5, 10, or more fold higher than that level found in a control sample or the average level of MICA or anti-MICA antibodies found in samples from a population of normal healthy mammals. A reduced level of MICA or anti-MICA antibodies can be 10, 20, 30, 50, 60, 70, 80, 90, 100, 150 or more percent lower than that level found in a control sample or the average level of MICA or anti-MICA antibodies found in samples from a population of normal healthy mammals. In some cases, a reduced level of MICA or anti-MICA antibodies can be 1, 2, 3, 4, 5, 10, 20, 50 or more fold lower than that level found in a control sample or the average level of MICA or anti-MICA antibodies found in samples from a population of normal healthy mammals.

In some cases, a reference chart can be used to determine whether or not a particular level of MICA or anti-MICA antibodies in a sample is reduced, normal, or elevated relative to a control sample or a larger population. For example, a reference chart can contain the normal range of MICA or anti-MICA antibodies found in healthy individuals of the same age, gestational age, ethnic background or general health as the individual in question. Using this reference chart, any level of MICA or anti-MICA antibodies measured in a sample can be classified as being an reduced, normal, or elevated relative to a control sample or a larger population.

Alternatively, or in addition, the level of MICA or anti-MICA antibodies in a biological sample can be “normalized” against one another or against the level of one or more additional biological markers. The values for the level of cell-associated MICA, sMICA or anti-MICA antibodies may be expressed as a ratio and the ratios may be compared to similar ratio obtained for a reference sample or population. That is, the levels of the additional marker can be evaluated in parallel with those of MICA or anti-MICA antibodies, either at the same time or on a separate occasion. The additional marker can serve as an internal control for sample preparation, handling and storage as well as day-to-day assay variability.

Once the relative level of MICA or anti-MICA antibodies in an individual relative to that of a reference sample has been calculated, the individual's relative risk for progression to malignancy can be assessed. Any statistical method known in the art for evaluating relative risk may be used, for example receiver operator characteristic curve analysis. The receiver operated characteristics (ROC) value describes the balance between the sensitivity (i.e., the number of hits detected) and the specificity (i.e., the accuracy) of a test. These two variables may also be considered positive predictive value and negative predictive value, and are correlated with diagnostic accuracy. The ROC curve shows the relationship of the probability of a positive test, given no disease, to the probability of a positive test, given disease. An ROC cutoff value is chosen to maximize diagnostic accuracy of the test in question. Following assessment of relative risk for progression, appropriate therapies, such as the administration of anti-MICA antibodies described above, as well as conventional cancer therapies can be initiated.

### Combination Therapy

In some embodiments compositions comprising two or more MICA modulators (e.g., inhibitors) are provided. In some embodiments the MICA modulators are monoclonal antibodies. Compositions comprising two or more anti-MICA antibodies or two or more anti-PDI, for example, ERp5, antibodies (or a combination of anti-MICA and anti-ERp5 antibodies) may be administered to persons or mammals suffering from, or predisposed to suffer from, cancer. In some embodiments compositions comprising two or more Erp5 modulators (e.g., inhibitors) are provided. In some embodiments the Erp5 modulators are monoclonal antibodies. One or more antibodies may also be administered with another therapeutic agent, such as a cytotoxic agent, or cancer chemotherapeutic. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In some embodiments the methods provided contemplate the administration of combinations, or “cocktails”, of different antibodies. Such antibody cocktails may have certain advantages inasmuch as they contain antibodies which exploit different effector mechanisms or combine directly cytotoxic antibodies with antibodies that rely on immune effector functionality. Such antibodies in combination may exhibit synergistic therapeutic effects. Useful antibodies can include antibodies that target the EGF receptor, e.g., Cetuximab (Erbitux™), antibodies that target VEGF, e.g., Bevacizumab (Avastin™) and antibodies that target Her-2, e.g., trastuzimab (Herceptin™)

A cytotoxic agent refers to a substance that inhibits or prevents the function of cells and/or causes destruction of cells. The term is intended to include radioactive isotopes (e.g., ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody disclosed herein.

In some embodiments, conventional cancer medicaments are administered with the compositions disclosed herein. Highly suitable agents include those agents that promote DNA-damage, e.g., double stranded breaks in cellular DNA, in cancer cells. Any form of DNA-damaging agent know to those of skill in the art can be used. DNA damage can typically be produced by radiation therapy and/or chemotherapy. Examples of radiation therapy include, without limitation, external radiation therapy and internal radiation therapy (also called brachytherapy). Energy sources for external radiation therapy include x-rays, gamma rays and particle beams; energy sources used in internal radiation include radioactive iodine (iodine¹²⁵ or iodine¹³¹), and from strontium⁸⁹, or radioisotopes of phosphorous, palladium, cesium, iridium, phosphate, or cobalt. Methods of administering radiation therapy are well know to those of skill in the art.

Examples of DNA-damaging chemotherapeutic agents include, without limitation, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane)

Other cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, caminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine), thalidomide, and lenalidomide (Revlimid®).

Other suitable chemotherapeutics include proteasome inhibiting agents. Proteasome inhibitors block the action of proteasomes, cellular complexes that degrade proteins, particularly those short-lived proteins that are involved in cell maintenance, growth, division, and cell death. Examples of proteasome inhibitors include bortezomib (Velcade®), lactacystin (AG Scientific, Inc., San Diego, Calif.), MG132 (Biomol International, Plymouth Meeting, Pa.) PS-519, eponemycin, epoxomycin, aclacinomycin A, the dipeptide benzamide, CVT-63417, and vinyl sulfone tripeptide proteasome inhibitors.

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; and anti-angiogenesis factors.

Prodrug refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, b-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

### EXAMPLES

### Example 1

### Methods and Materials

Clinical Protocols. Sera, lymphocytes, and tumor samples were obtained from patients enrolled on Institutional Review Board/Food and Drug Administration/Recombinant DNA Advisory Committee-approved Dana-Farber Partners Cancer Care clinical protocols. The Phase I trial entailed vaccination with lethally irradiated, autologous tumor cells engineered to secrete GM-CSF in advanced melanoma and non-small cell lung carcinoma patients or patients with acute myeloid leukemia/advanced myelodysplasia and ovarian carcinoma. The Phase I trial of the fully human anti-CTLA-4 blocking monoclonal antibody (Ipilumimiab®) in previously vaccinated melanoma and ovarian carcinoma patients has been described.
cDNA library construction and screening. The K008 and M34 melanoma cDNA expression libraries were generated and screened as follows. Total RNA was isolated from the melanoma cell line K008 by using guanidine isothiocyanate, and the mRNA was selected with two rounds of oligo(dT) cellulose. A cDNA expression library was constructed in the Lambda Zap vector by using a commercial cDNA library kit (Stratagene) according to the manufacturer's procedures. Plaques (1×10⁶) were screened with precleared (against Escherichia coli and λ phage lysates) postvaccination sera from patient MEL15 after MDX-010 infusion at a 1:1,000 dilution in TBS/0.1% Tween-20/2% nonfat dried milk (NFDM). Positive plaques were detected with an alkaline phosphatase-conjugated goat anti-human IgG antibody (Jackson ImmunoResearch) diluted 1:2,000 in TBST (50 mM Tris/138 mM NaCl/2.7 mM KCl/0.05% Tween 20, pH 8.0). Reactive clones were plaque-purified, and the excised phagemids were sequenced.

A cDNA expression library was generated from RENCA cells as follows. Total RNA was isolated using guanidine isothiacyanate, mRNA purified over oligo-dT cellulose columns, and cDNA synthesized with Superscript II Reverse Transcriptase (RT, Invitrogen). The cDNA was cloned into the Lambda Zap vector and the library screened according to the manufacturer's instructions (ZAP-cDNA Gigapack III Gold cloning and picoBlue Immunoscreening kits, Stratagene). Sera were pooled from five vaccinated mice and pre-absorbed against E. coli lysed with non-recombinant phage. 1×106 plaques were plated and screened with sera diluted 1:300 in TBS/0.1% Tween-20/2% nonfat dried milk (NFDM) and 0.01% (w/v) sodium azide. Reactive clones were detected with a goat anti-mouse pan IgG antibody conjugated to alkaline phosphatase (Jackson ImmunoResearch laboratories) and plaque purified through sequential re-platings. Plasmid DNA from positive clones was isolated, and the cDNA inserts were sequenced (Molecular Biology Core Facility, Dana-Farber Cancer Institute) and analyzed with the GenBank BLASTN and BLASTX algorithms (National Center for Biotechnology Information) and the Cancer Immunome Database (www2.licr.org/cancerimmunomeDB).

Serology. Anti-MICA antibodies and sMICA levels were measured with ELISAs using recombinant MICA protein (ProSpec-Tany TechnoGene Inc.) and anti-human MICA monoclonal antibodies (R&D Systems). The lower limits of the assay were 90 pg/ml. The Wilcoxon rank sum test with no adjustments for multiple comparisons was used to analyze the differences among the normal donors, MGUS, and MM patients. For complement assays, 293T and 293-T-MICA cells, generated by retroviral mediated gene transfer were incubated with patient sera and human complement (Sigma-Aldrich) and lysis quantified using MTT (Roche Diagnostic GmbH).

Anti-PDI antibodies were measured by coating ELISA plates (Nunc) overnight at 4° C. with 1-5 μg/ml of histidine-tagged recombinant human PDI protein (ProSpec-TechnoGene) or control histidine peptide (New England peptide) dissolved in a carbonate buffer, pH 9.6. Next, the wells were blocked overnight at 4° C. with 2% NFDM/PBS, washed, and then incubated in triplicate with 100 μl of patient sera diluted 1:100 in 2% NFDM/PBS overnight at 4° C. A goat anti-human IgG conjugated to alkaline-phosphatase (Jackson) was added at room temperature and the plate developed with pNPP substrate (Sigma). The absorbance of PDI minus control histidine peptide at 405 nm was determined.

Anti-ERp5 antibodies were assayed by coating the ELISA plates with 1 μg/ml of gluthathione S-transferase-ERp5 (GST-ERp5) (Abnova) or GST recombinant protein produced with the PGEX 5X-3 vector (Amersham Pharmacia). After blocking overnight with 2% NFDM/PBS at 4° C., the wells were washed in PBST and incubated in triplicate overnight at 4° C. with patient sera diluted 1:50 or a rabbit polyclonal anti-human ERp5 antibody diluted 1:500 (Axxora) as a positive control. A goat anti-human IgG conjugated to alkaline-phosphatase (Jackson) was added at room temperature and the plate developed with pNPP substrate (Sigma). The absorbance of ERp5 minus GST at 405 nm was determined.

Immunoblotting. Antibodies used for immunoblotting were: anti-phospho-ATM (ser 1981), anti-ATM, anti-phospho-ATR (ser 428), anti-ATR, anti-phospho-chk-1 (ser 280) and anti-phospho-chk-2 (thr 68) (Cell Signaling, Beverly, Mass.). Immunoblotting was performed according to standard methods. In some experiments, U226 cells were transiently transfected with expression plasmids encoding human chk-1 shRNA (5′-CAACTTGCTGTGAATAGAAT-3′), chk-2 shRNA, or ATM shRNA (Upstate cell signaling, Lake Placid, N.Y.) according to the manufacturer's instructions. The efficiency of gene knockdown was 50%-90% as assessed by immunoblotting.
Flow Cytometry. PBMCs and bone marrow aspirates were processed by Ficoll-Hypaque (Pharmacia) density-gradient centrifugation. PBMCs from healthy donors or patients were incubated for 48 hours in patient or control sera (10%) in complete media and stained with PE-conjugated anti-NKG2D mAb (Pharmingen), FITC-conjugated anti-CD3 mAb (BD-Biosciences Pharmingen), and either PC5-conjugated anti-CD8 mAb or PC5-conjugated anti-CD56 mAb (Beckman-Coulter). In Examples 9-11, antibodies used, in addition to the above, were: FITC-conjugated anti-CD 138, PE-conjugated anti-MICA mAb (R&D Systems), and anti-ERp5 (Axxora Platform) followed by FITC-conjugated anti-rabbit (Southern Biotech). Cells were analyzed with a FW501 flow cytometer (Beckman-Coulter) and FlowJo software (Tree Star). Cells were gated for CD3⁺CD8⁺T cells and CD3⁻CD56⁺NK cells. RENCA cells (5×10⁵) were incubated with sera (1:100 dilution) from naïve or vaccinated mice or an IgG isotype control for 3 hours at room temperature, washed, and then stained with a secondary, PE-conjugated goat anti-mouse IgG (Jackson ImmunoResearch). Cells were analyzed with a FACScan cytometer (Becton Dickinson).
Cellular Assays. Donor peripheral blood mononuclear cells (PBMCs) were incubated in 10% patient sera as described above, and NK cells, isolated by magnetic cell sorting (Miltenyl Biotech), were tested in four-hour lysis assays against ⁵¹Cr-labeled K562 target cells. The spontaneous release in all assays was less than 20% of the maximum. Dendritic cells (generated from adherent PBMCs with GM-CSF and IL-4) were co-cultured with opsonized tumor cells (1:1 ratio) for 20 hours, matured with LPS (Sigma-Aldrich) for 24 hours, and then used to stimulate autologous CD8+ T cells, isolated by magnetic cell sorting, for seven days. Antigen specific IFN-γ production was then determined by ELISPOT. For enzyme-linked immunospot (ELISPOT) analysis, ImmunoSpotplates (Cellular Technology) were coated overnight at 37° C. with 10 μg/ml anti-IFN-γ mAb (Mabtech). Cells harvested from metastases were mixed 1:1 with peripheral blood mononuclear cells (as a source of antigen-presenting cells) and plated at 2×10⁵ cells per well with 1 μg/ml of HLA-A2-restricted peptides. The peptides were derived from MART-1 (M27; AAGIGILTV), gp100 (G154; KTWGQYWQV) and tyrosinase (368D: YMDGTMSQV). After 24 h at 37° C., the wells were washed and then incubated with 1 μg/ml biotin-conjugated anti-IFN-γ mAb (Mabtech) followed by streptavidin-alkaline phosphatase (Mabtech). Spots were developed by using BCIP/NBT as a color development substrate and counted with an ImmunoSpot microplate reader (Cellular Technology, Cleveland).
Multiple Myeloma Cell Lines. U226, RPMI, and MM-1R cells were obtained from the American Type Culture Collection (Manassas, Va.), and MM-1S cells were a gift of Dr. Steven Rosen (Northwestern University). Multiple myeloma cell lines were cultured in complete media (RPMI 1640, 10% heat-inactivated fetal calf serum, penicillin, and streptomycin), and in some experiments treated with 5 to 20 nM Bortezomib (Millennium Pharmaceuticals), 10 μg/ml aphidicolin (Sigma-Aldrich), or 250 μg/ml dexamethasone for up to 16 hours. U226-MICA cells were generated with retroviral mediated gene transfer according to standard methods.
Pathology. Zenker fixed, decalcified bone marrow tissue microarrays were embedded in paraffin and sectioned at 5 μm thick. The microarrays were treated for antigen retrieval with a pressure cooker for 20 min and then incubated with 5 μg/ml of primary antibodies or a corresponding IgG fraction of pre-immune serum in 3% BSA/PBS blocking solution for 16 hours at 4° C. Antibodies used for immunohistochemistry were: rabbit anti-phospho-chk-2 (thr 68) Ab (Cell Signaling Technology, Danvers, Mass.), mouse anti-ERp5 Ab (Axxora Platform, San Diego, Calif.), and mouse anti-MICA Ab (Pharmingen, San Diego, Calif.). The primary antibodies were then visualized with the corresponding secondary biotinylated antibody and the streptavidin-peroxidase complex from Vector Labs (Burlingame, Calif.).
Murine tumor model. RENCA (renal cell carcinoma) cells were cultured in Dulbecco's Modified Eagle's Medium containing 10% (v/v) inactivated fetal calf serum, 100 units/ml penicillin/streptomycin, 1 mM non-essential amino acids, and 10 mM HEPES buffer (pH 7.4). Syngeneic, female BALB/c mice from 8-12 weeks of age were obtained from Taconic Farms. Animals were immunized subcutaneously on the abdominal wall with 5×105 irradiated (35 Gy), GM-CSF-secreting or wild type Renca cells at weekly intervals. Sera were obtained at varying times during vaccination by eye bleeding. All mouse experiments were conducted under a protocol approved by the AAALAC-accredited Dana-Farber Cancer Institute IACUC.
Tumor antigen expression. TRIZOL (GIBCO/BRL) was used to isolate total RNA from tumor cells and normal tissues. 10 μg total RNA was electrophoresed through an agarose formaldehyde gel in MOPS running buffer, transferred to nylon membranes (Hybond-XL, Amersham Biosciences), and cross-linked with a UV Stratalinker 2400 (Stratagene). ³²P-labeled (NEN/Perkin Elmer Life Sciences) probes ranging from 500 to 1500 nucleotides were prepared with 25 ng of template DNA and the Prime-It II Random Primer Labeling Kit (Stratagene). The 18S ribosomal RNA was used as a loading control. Hybridizations were performed overnight at 68° C., and then the filters were extensively washed and developed.
cDNA library construction and screening. A cDNA expression library was generated from RENCA cells according to standard methods. In brief, total RNA was isolated using guanidine isothiacyanate, mRNA purified over oligo-dT cellulose columns, and cDNA synthesized with Superscript II Reverse Transcriptase (RT, Invitrogen). The cDNA was cloned into the Lambda Zap vector and the library screened according to the manufacturer's instructions (ZAP-cDNA Gigapack III Gold cloning and picoBlue Immunoscreening kits, Stratagene). Sera were pooled from five vaccinated mice and pre-absorbed against E. coli lysed with non-recombinant phage. 1×106 plaques were plated and screened with sera diluted 1:300 in TBS/0.1% Tween-20/2% nonfat dried milk (NFDM) and 0.01% (w/v) sodium azide. Reactive clones were detected with a goat anti-mouse pan IgG antibody conjugated to alkaline phosphatase (Jackson ImmunoResearch laboratories) and plaque purified through sequential re-platings. Plasmid DNA from to positive clones was isolated, and the cDNA inserts were sequenced (Molecular Biology Core Facility, Dana-Farber Cancer Institute) and analyzed with the GenBank BLASTN and BLASTX algorithms (National Center for Biotechnology Information) and the Cancer Immunome Database (www2.licr.org/cancerimmunomeDB).

### Example 2

### Identification of a Serological Response to MICA in an Advanced Melanoma Patient Treated with CTLA-4 Antibody Blockade

Clinical course. MEL15 is a 48 years old female who had a primary melanoma removed in 2000. Four years later, she developed abdominal pain and was found to harbor multiple lung and pleural-based nodules, primarily left-sided, that were biopsy proved as metastatic disease. She underwent thoracotomy on protocol to harvest tissue for autologous, GM-CSF secreting tumor cell vaccine manufacture and received six immunizations (the first three at weekly intervals and the last three at every two weeks) during May and June 2004. Vaccination evoked strong local reactions and delayed-type hypersensitivity responses to injections of irradiated, autologous, unmodified melanoma cells, but thoracic CT scans at re-staging disclosed slightly enlarged pulmonary metastases.

In August 2004, MEL15 complained of significant left-sided chest pain radiating to the left shoulder and neck, likely referred from the pleural metastases, and narcotic analgesia was instituted. Treatment on protocol with the fully human anti-CTLA-4 monoclonal antibody (MDX-010) at 3 mg/kg was begun, and one month later MEL15 reported a marked improvement in the referred pain. Repeat CT scans in September 2004, two months after the initiation of CTLA-4 antibody blockade, demonstrated a mixed response with a slight increase in some lung masses (less than 10%), but a reduction in the pleural-based lesions. MEL15 received additional MDX-010 infusions at two-month intervals (for a total of nine treatments as of March 2006), with complete resolution of the pain and requirement for analgesia. Her lung disease steadily improved, with the most recent CT scans documenting a >50% reduction in the size of all lesions. Toxicities of therapy were limited to a mild erythematous rash, reflecting the development of lymphocyte infiltrates in the superficial dermis with some T cell apposition to normal melanocytes.

Serologic Response to MICA. Cancer antigens associated with the reduction in MEL15′ s tumor burden were identified by screening melanoma cDNA expression libraries with MEL15 sera. Sera obtained from MEL15 after MDX-010 infusion was used to screen two melanoma cDNA expression libraries constructed from heavily infiltrated metastases of patients achieving long-term responses to autologous, GM-CSF secreting tumor vaccines. Library construction and screening were performed according to the methods described in Example 1.

Table 1 shows the 16 gene products that were identified. Pre-VAX, Post-VAX and Post-MDX-010 refer to sera obtained from MEL15 prior to receiving autologous GM-CSF secreting tumor cell vaccine, after receiving autologous GM-CSF secreting tumor cell vaccine, and after receiving MDX-010 respectively. MICA was detected in both libraries and was selected for further analysis.

TABLE 1
Targets identified in melanoma cDNA expression libraries using MEL15 sera

| Pre-VAX | | Post-VAX | | Post-MDX-010 | |
|---|---|---|---|---|---|
| | GenBank ® | | GenBank ® | | GenBank ® |
| | Accession | | Accession | | Accession |
| Antigen | number | Antigen | number | Antigen | Number |
| K008 Library | | K008 Library | | K008 Library | |
| Iron-transport | BC009642 | Stearoyl CoA | AF97514 | Galectin-3 | AF031425 |
| regulator 5 | | desaturase | | | |
| Pleckstrin | BC01829 | Macrophage | BC022414 | Actin-gamma | BC018774 |
| homology- | | migration | | | |
| like domain | | inhibitory | | | |
| family | | factor | | | |
| member 2 | | | | | |
| Cathepsin B | BC010240 | Calumerin | BC013383 | Stearoyl CoA desaturase | AF97514 |
| LEREP04 | AF109366 | KIAA1049 | BC009349 | Macrophage migration | BC022414 |
| | | | | inhibitory factor | |
| | | | | Ribosomal protein synthase | BC016378 |
| | | | | II | |
| | | | | Annexin 2A | BC013843 |
| | | | | NADH dehydroxygenase | AL136962 |
| | | | | subunit 3 | |
| | | | | CD63 | BT020138 |
| | | | | IFN-induced transmembrane | BC070243 |
| | | | | protein 3 | |
| | | | | F-box protein 7 | BC041004 |
| | | | | Formin-like 2 isoform A-D | NM052905 |
| | | | | MICA | AF264741 |
| | | | | M34 Library | |
| | | | | Tubulin alpha 6 | BC013383 |
| | | | | Torsin A-interacting protein 1 | NM015602 |
| | | | | Mannosyl transferase | AF007906 |
| | | | | ATP synthase | BC001178 |
| | | | | (mitochondrial complex, | |
| | | | | subunit F6) | |
| | | | | MICA | AF264741 |

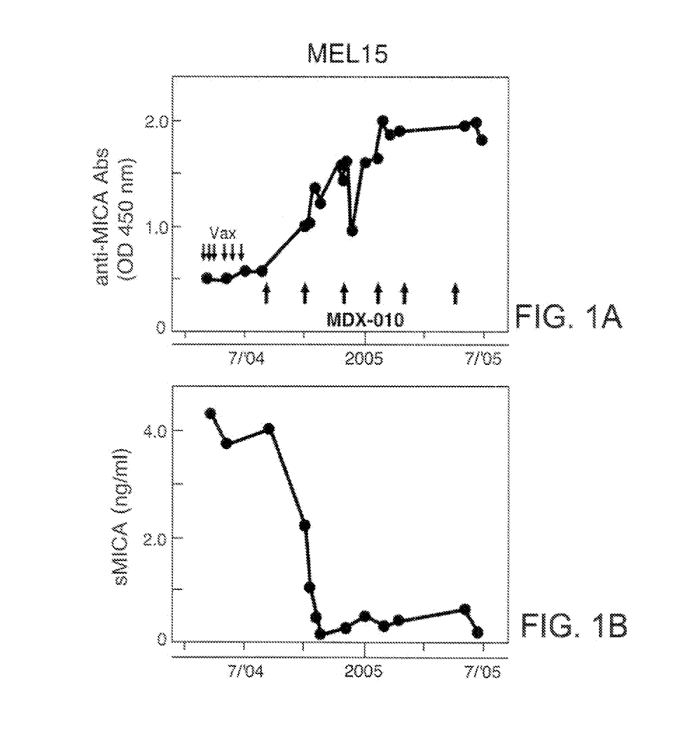

The humoral response to MICA was analyzed by an ELISA with recombinant MICA protein as described in Example 1. Longitudinal sera samples from MEL15 were diluted 1:500. The results of this time course analysis are shown in FIG. 1A. Downward arrows denote tumor cell vaccinations and upward arrows depict infusions of MDX-010. Unexpectedly, MEL15 harbored anti-MICA antibodies prior to vaccination, likely indicative of a nascent host reaction, as sera from 20 healthy controls failed to recognize the protein. Vaccination with autologous GM-CSF secreting tumor cells produced a modest increase in anti-MICA antibody titers; infusion of MDX-010 produced elevated levels of anti-MICA antibodies which were sustained with continued treatment. Additional analysis revealed that IgG2 antibodies constituted the dominant anti-MICA immunoglobulin subclass.

Soluble MICA (sMICA) levels were also analyzed. As shown in FIG. 1B, sMICA levels were elevated upon study entry and during vaccination; CTLA-4 antibody blockade resulted in a sharp decrease in sMICA that was temporally linked to the rise in anti-MICA antibodies (FIG. 1A). The reduction in sMICA was confirmed with immunoblotting analysis of MEL15 sera using an anti-MICA monoclonal antibody.

### Example 3

### The Effect of Therapy Induced Anti-MICA Antibodies on Innate Antitumor Immunity

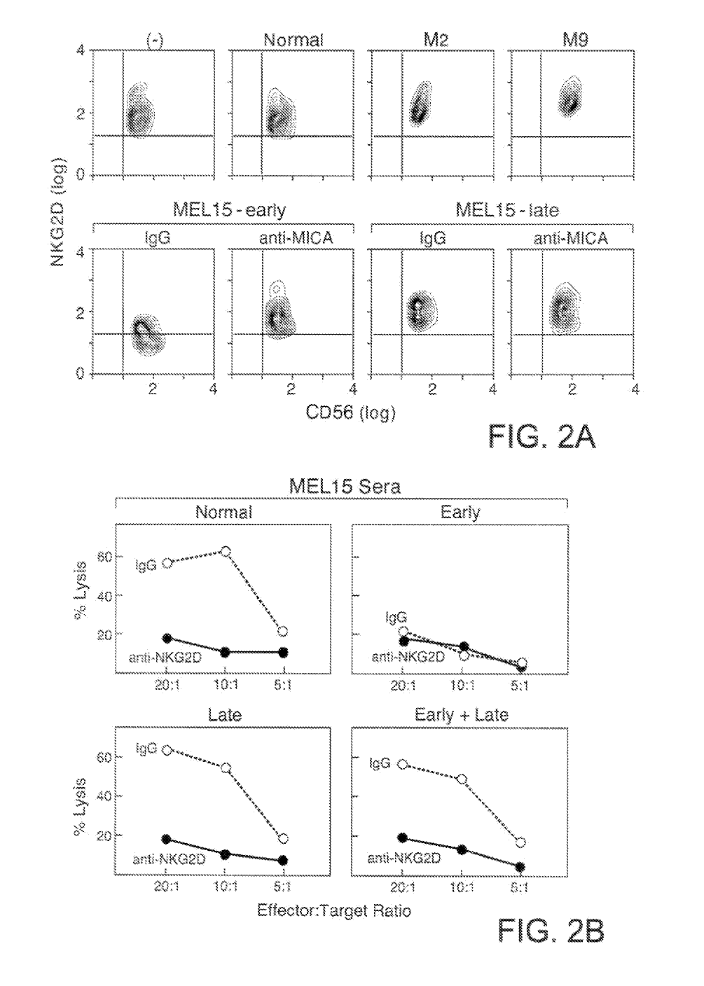

The effect of anti-MICA antibodies on NKG2D expression on gated NK cells was analyzed. CD56+,CD3−NK cells were purified from healthy donors, cultured for 48 hours in various sera, and analyzed by flow cytometry (FIG. 2A). Serum samples were obtained from a healthy donor, vaccinated melanoma patients without sMICA (M2 and M9), and MEL15 during immunization or after CTLA-4 antibody blockade. Anti-MICA monoclonal antibodies or isotype controls were added where indicated. Sera from normal donors or vaccinated melanoma patients (M2 and M9) without detectable sMICA (limits of detection 90 pg/ml) did not alter NKG2D expression. However, sera obtained from MEL15 during vaccination (“MEL15-early”) diminished NKG2D surface levels, whereas sera collected after MDX-010 infusion (“MEL15-late”) did not. The addition of anti-MICA monoclonal antibodies to MEL15 early sera blocked the decrease in NKG2D expression, suggesting that sMICA and not TGF-13 (or other factors) was the dominant suppressive mechanism. In accordance with this finding, late MEL15 sera also antagonized the NKG2D down-regulation triggered with early sera.

The ability of NK cells to lyse target cells in the presence of antiMICA antibodies was also evaluated. Healthy donor PBMCs were incubated in normal or MEL15 sera for 48 hours and washed; NK cells were then purified with magnetic bead selection and tested for lytic activity against ⁵¹Cr-labeled K562 targets. The NKG2D-dependent lysis was determined with the addition of the mAb 1D11 (anti-NKG2D) or isotype control (IgG).

As shown in FIG. 2B, donor NK cells exposed to normal sera efficiently lysed K562 cells; lysis was substantially blocked with anti-NKG2D antibodies, indicating a major role for NKG2D in target cell recognition. However, NK cells incubated in MEL15 early sera showed impaired K562 killing, while NK cells cultured in MEL15 late sera manifested NKG2D-dependent lysis that was comparable to healthy controls. NK cells incubated in a mixture of MEL15 early and late sera also displayed robust NKG2D-dependent killing, illustrating that high titer anti-MICA antibodies neutralized the deleterious effects of sMICA.

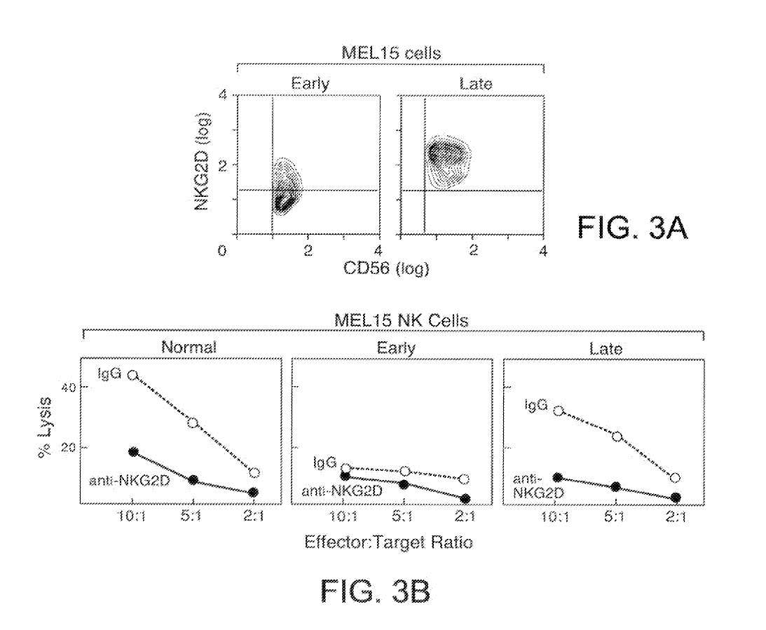

NKG2D levels on CD56+ cells obtained from MEL15 were also analyzed. PBMCs were obtained from MEL15 during vaccination or after CTLA-4 blockade, and NKG2D expression on gated NK cells was determined with flow cytometry. As shown in FIG. 3A, NKG2D levels were reduced on CD56+ cells obtained from MEL15 during vaccination (“early”) as compared to those collected after CTLA-4 antibody blockade (“late”).

The ability of MEL15 NK cells to lyse target cells in the presence of antiMICA antibodies was also evaluated. Magnetic bead-purified healthy donor or MEL15 NK cells obtained at different times were tested for lytic activity against ⁵¹Cr-labeled K562 targets. As shown in FIG. 3B, MEL15 NK cells collected during vaccination displayed decreased killing (“early”), whereas NK cells obtained after MDX-010 infusion (“late”) mediated NKG2D-dependent lysis at levels that were nearly equivalent to normal controls.

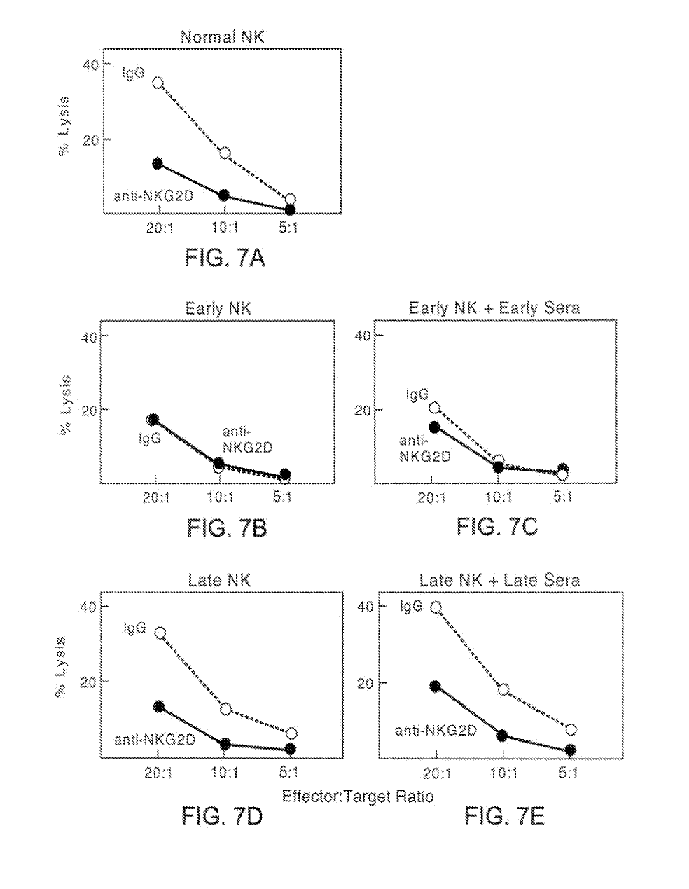

The therapy-induced anti-MICA antibodies did not block NK cell lysis of K562 cells. Magnetic bead-purified healthy donor or MEL15 NK cells obtained at different times were tested for lytic activity against 51Cr-labeled K562 targets. Autologous sera were added to the lytic assay where indicated. As shown in FIGS. 7A-F, MEL15 CD56+ cells collected after CTLA-4 blockade efficiently killed K562 cells in the presence of late sera (Panels 7D and 7E) indicating that high titer anti-MICA antibodies did not interfere with target cell lysis.

### Example 4

### The Effect of Therapy Induced Anti-MICA Antibodies on Adaptive Antitumor Immunity

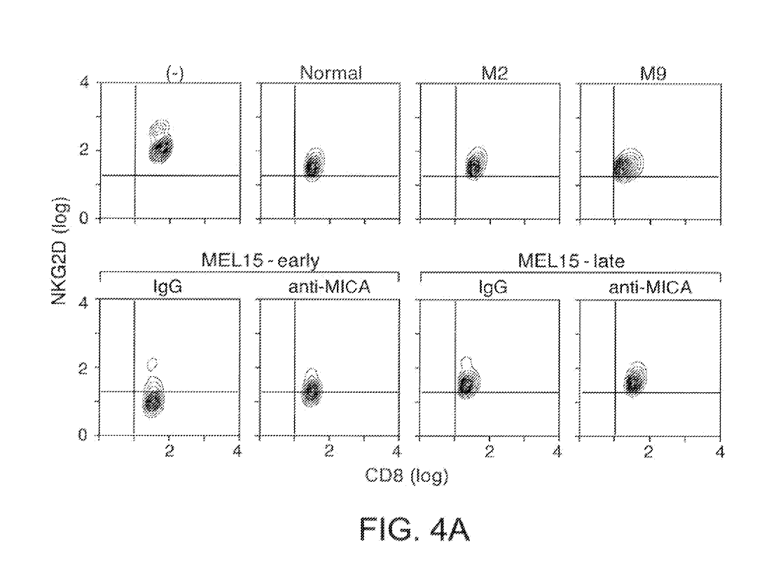

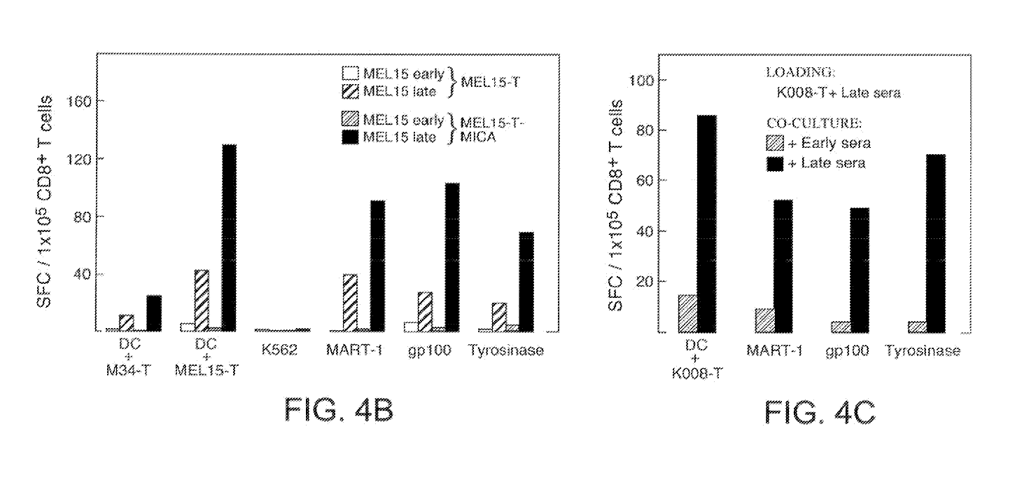

The effect of therapy induced anti-MICA antibodies on adaptive tumor immunity was analyzed. First, the ability of sMICA from MEL15 to inhibit CD8+ T lymphocyte cytotoxicity through down-regulation of NKG2D was assayed. Normal donor PBMCs were incubated for 48 hours in various sera, and NKG2D expression on gated CD8+ T cells (CD8+, CD3+) was determined with flow cytometry (FIG. 4A). Sera samples were obtained from a healthy donor, vaccinated melanoma patients without sMICA (M2 and M9), and MEL15 during immunization or after CTLA-4 antibody blockade. Anti-MICA monoclonal antibodies or isotype controls were added where indicated.

Compared to freshly isolated cells, NKG2D expression was modestly decreased on CD8⁺T lymphocytes incubated in sera from healthy donors (“normal”) or vaccinated melanoma patients (“M2” and “M9”) without detectable sMICA. However, sera obtained from MEL15 during vaccination (“MEL15-early”) evoked significantly greater reductions in NKG2D levels, while sera collected after MDX-010 infusion (“MEL15-late”) proved equivalent to controls. The addition of anti-MICA monoclonal antibodies or MEL15 late sera to MEL15 early sera blocked the decrease in NKG2D expression, establishing sMICA as the primary suppressive factor.

The ability of therapy-induced anti-MICA antibodies to enhance cross-presentation was evaluated in an autologous melanoma cell line (MEL15-T) from the pulmonary metastasis resected for vaccine manufacture. MEL15-T cells with stable, high-level MICA expression (MEL15-T-MICA) were generated by retroviral mediated gene transfer; while MEL15-T cells displayed only low levels of MICA during routine culture, gamma-irradiation augmented expression consistent with previous work linking MICA to the DNA damage response.

Since MEL15 is HLA-A2⁻, the ability of HLA-A2⁺ healthy donors to cross-present opsonized MEL15-T and MEL15-T-MICA cells was assayed. Dendritic cells were generated from HLA-A2⁺ normal donors by culturing peripheral blood monocytes in GM-CSF and IL-4. The expanded dendritic cells were pulsed with loaded with MEL15 early or late sera-coated MEL15-T or MEL15-T-MICA tumor cells, matured with LPS, and used to stimulate purified donor CD8⁺T cells for seven days. Melanoma-specific IFN-γ production was measured by ELISPOT against the indicated targets (FIG. 4B).

As shown in FIG. 4B, MEL15 sera obtained after MDX-010 infusion (“MEL15 late”) mediated more efficient MICA-dependent cross-presentation of melanoma antigens than MEL15 sera collected during vaccination (“MEL15 early”). This resulted in HLA-A2-restricted CD8⁺T cell responses to peptides derived from MART-1, gp100, and tyrosinase, and low-level recognition of dendritic cells loaded with MEL15 late sera-coated M34-T melanoma cells (HLA-A2⁺). MEL15 sera obtained after CTLA-4 blockade also enhanced the cross-presentation of MEL15-T cells, albeit to a lesser extent. Control experiments indicated that only minimal reactivity was induced against K562 cells or dendritic cells loaded with unopsonized tumor cells and that no reactivity was induced against unpulsed dendritic cells.

This cross-presentation scheme was then employed to evaluate whether the sMICA-induced down-regulation of NKG2D impaired the generation of tumor-specific CD8⁺T lymphocytes. For these studies, K008-T melanoma cells, which constitutively express high levels of MICA, were opsonized with MEL15 sera obtained after CTLA-4 blockade and loaded onto HLA-A2.1+ dendritic cells. After maturation with LPS, the dendritic cells and CD8⁺T cells were co-cultured in the presence of either MEL15 sera obtained during vaccination (“early sera”) or MEL15 sera collected after CTLA-4 blockade (“late sera”). IFN-γ production was measured by ELISPOT against the indicated targets.

As shown in FIG. 4C, MEL15 early, but not late sera markedly inhibited the development of CD8⁺T cell responses to opsonized K008-T cells and melanosomal differentiation antigens, illustrating the potent suppressive effects of sMICA on adaptive cellular immunity.

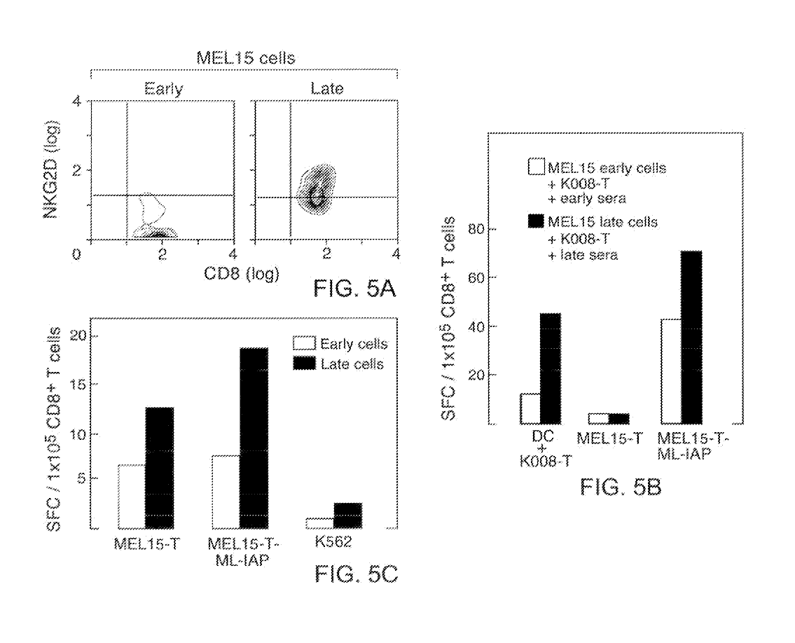

As shown in FIG. 5, immunotherapy restored protective anti-tumor innate responses and enhanced cross-presentation in MEL15. PBMCs were obtained from MEL15 during vaccination or after CTLA-4 blockade, and NKG2D expression on gated CD8+ T cells was determined with flow cytometry. NKG2D levels were substantially decreased on CD8⁺T cells collected from MEL15 during vaccination (“early”) relative to those obtained after MDX-010 infusion (“late”) (FIG. 5A). Moreover, MEL15 samples collected after CTLA-4 blockade manifested much greater cross-presentation of K008-T cells compared to samples obtained during vaccination (FIG. 5B), resulting in enhanced specific reactions against, melanoma inhibitor of apoptosis protein (ML-IAP), a previously characterized tumor rejection antigen. For the experiments shown in FIG. 5B, PBMCs were obtained from MEL15 during vaccination or after CTLA-4 blockade and used to generate dendritic cells. These were loaded with K008-T melanoma cells coated in early or late MEL15 sera and then matured with LPS. Purified CD8+ T cells from the same time points were then stimulated in vitro with the respective tumor-loaded dendritic cells for seven days. IFN-γ production was measured by ELISPOT against the indicated targets. Augmented CD8⁺ T cell responses following MDX-010 infusion were also evident without in vitro stimulation (FIG. 5C). For the experiment shown in FIG. 5C, MEL15 CD8+ T cells were purified from PBMCs collected during vaccination (“MEL15 early cells”) or after CTLA-4 blockade (“MEL15 late cells”) and tested for IFN-γ production against the indicated targets without prior in vitro stimulation.

To test the hypothesis that CTLA-4 blockade evoked a diversification of antigen recognition that accounted for increased cross-presentation and CD8+ T cell function in MEL15, the K008 melanoma cDNA expression library was screened with MEL15 sera obtained at study enrollment and after vaccination, but before MDX-010 administration. As shown in Table 1 (see example 1, above) only 4 targets were identified with MEL15 sera obtained at study enrollment (pre-vax) and after vaccination (post-vax), in contrast to the 12 gene products identified in this library with sera collected after CTLA-4 blockade (post-MDX-010). The greater number of antigens that elicited IgG antibody responses after MDX-010 infusion is consistent with a spreading of T cell reactivity.

### Example 5

### Vaccine-Induced Anti-MICA Antibodies

The ability of vaccination alone, in the absence of CTLA-4 blockade, to trigger the development of anti-MICA humoral immunity was evaluated in 14 additional metastatic melanoma or non-small cell lung carcinoma patients who were immunized with irradiated, autologous, GM-CSF secreting tumor cells.

Upon study entry, 10 patients harbored anti-MICA antibodies, and nine of these manifested circulating sMICA. Longitudinal analysis disclosed that vaccination augmented anti-MICA antibody titers that were temporally associated with decreases in sMICA in three cases, each of whom demonstrated pathologic and/or clinical evidence of anti-tumor activity. sMICA levels remained constant or rose in those subjects that did not display increased anti-MICA antibodies.

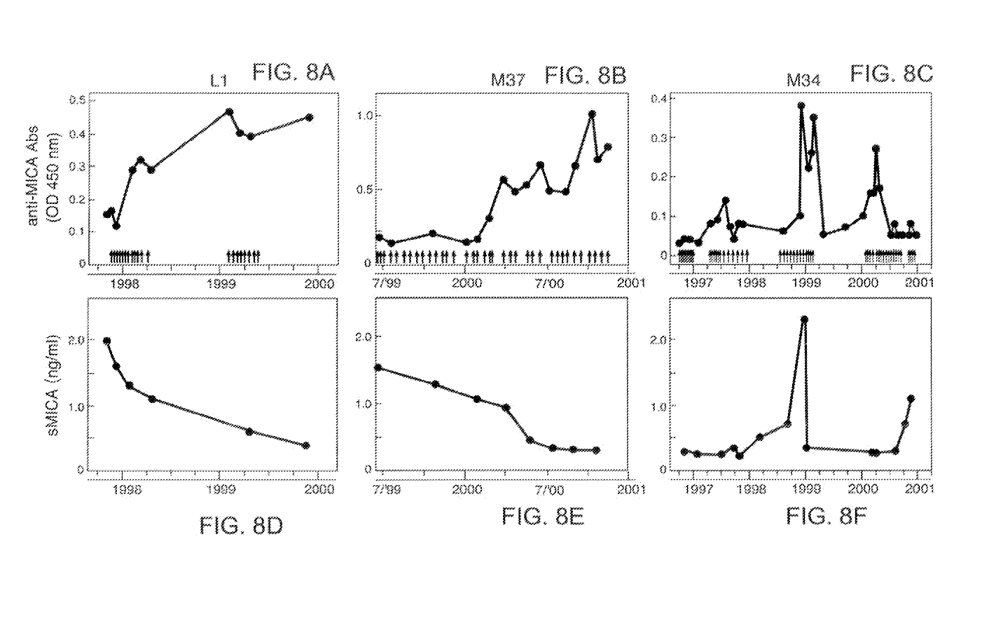

The levels of anti-MICA antibodies and the corresponding levels of sMICA in the three patients who demonstrated pathologic and/or clinical evidence of anti-tumor activity are shown in FIG. 8. The top panels (FIGS. 8A, 8B and 8C show levels of anti-MICA antibodies in longitudinal sera samples from vaccinated NSCLC patient L1 and vaccinated melanoma patients M37 and M34. For MICA analysis, samples were diluted 1:500 and analyzed by ELISA with recombinant MICA protein. Reactivity was determined with a pan-IgG secondary. sMICA levels, measured with a sandwich ELISA are shown in the lower panels (FIGS. 8D, 8C and 8E) Upward arrows depict tumor cell vaccinations. Patient L1 exhibited disseminated, progressive non-small cell lung carcinoma upon study enrollment, but vaccination stimulated dense T and B cell infiltrates in metastatic lesions and disease stabilization for nearly two years (survival of 31 months). Patient M37 entered study with visceral melanoma metastases, which were completely resected for vaccine manufacture. He was immunized for two years and remains disease-free six years after enrollment without further therapy. Patient M34 presented with visceral melanoma metastases and achieved a partial clinical response to an initial course of immunization. Subsequent isolated recurrences were processed into additional vaccines, whereas the patient succumbed to progressive disease 4.5 years after study entry.

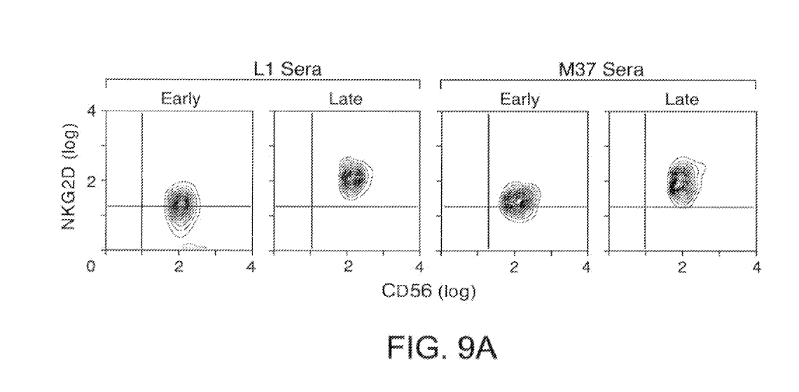

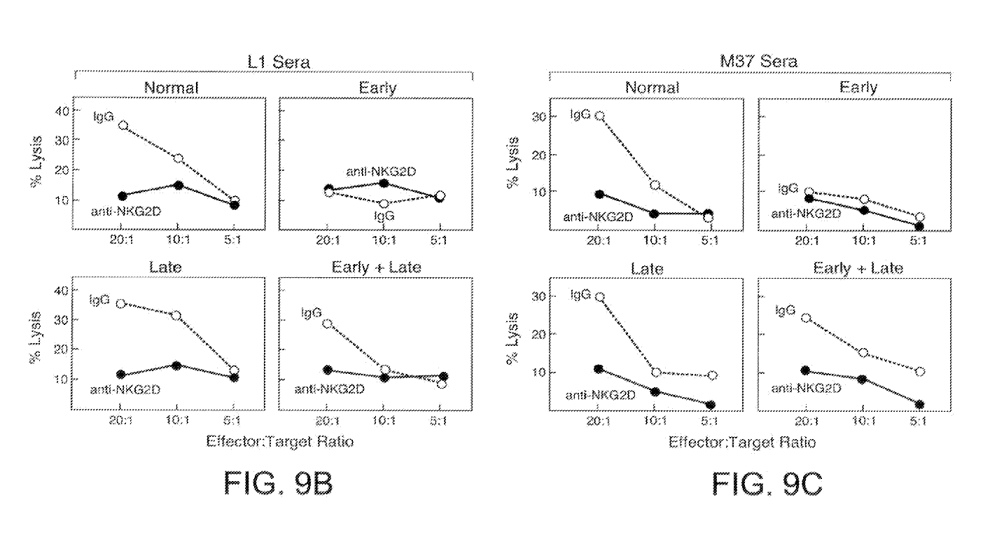

The effect of the vaccine-induced anti-MICA antibodies and decreased sMICA on both innate and adaptive antitumor immunity was explored in these three patients and was shown to be similar to that observed for patient MEL15. In particular, pre-vaccination sera down-regulated NKG2D levels and inhibited lytic activity of purified CD56⁺ cells from healthy donors, whereas late sera antagonized these suppressive effects. As shown in FIG. 9, vaccine-induced anti-MICA antibodies antagonized sMICA suppression of innate immune responses. For the experiment in FIG. 9A, normal donor PBMCs were incubated for 48 hours in sera, and NKG2D expression on gated NK cells was determined with flow cytometry. Sera samples were obtained before vaccination (“early”) and at the end of therapy (“late”). For the experiment in FIG. 9B, healthy donor PBMCs were incubated in L1 pre-(“early”) and post-vaccination (“late”) sera for 48 hours and washed; NK cells were then purified with magnetic bead selection and tested for lytic activity. For the experiment in FIG. 9C, NK cells were evaluated with M37 pre-(“early”) and post-vaccination (“late”) sera as above.

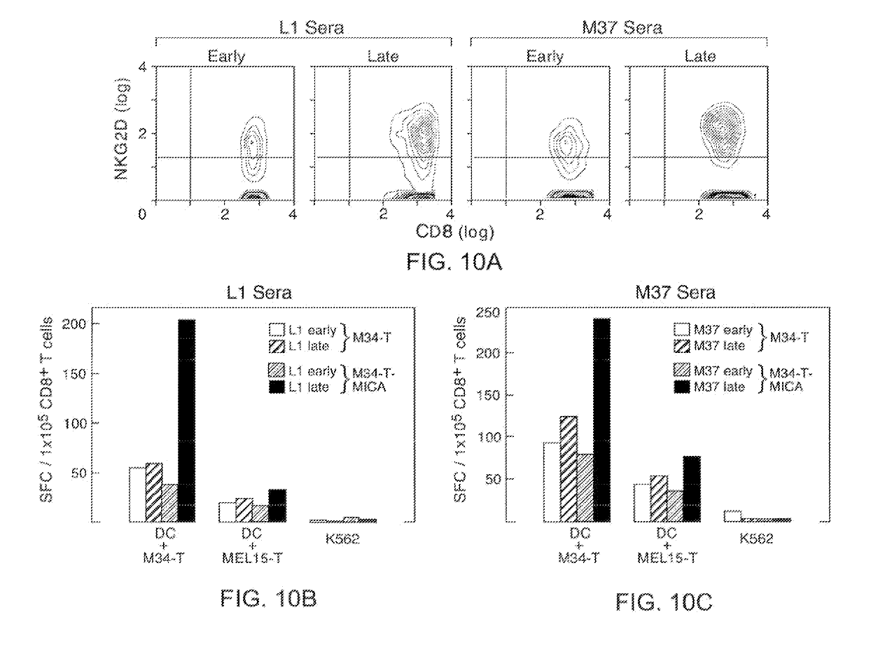

Further, early sera diminished NKG2D levels on healthy donor CD8⁺T cells, while post-vaccination sera promoted the efficient cross-presentation of MICA expressing melanoma cells. As shown in FIG. 10, vaccine-induced anti-MICA antibodies antagonized sMICA suppression of adaptive immune responses and enhanced MICA-dependent cross-presentation. For the experiment in FIG. 10A, normal donor PBMCs were incubated for 48 hours in L1 and M37 sera, and NKG2D expression on gated CD8+ T cells was determined with flow cytometry. For the experiment in FIG. 10B, the effects of L1 sera on the cross-presentation of M34-T and M34T-MICA cells was determined as in Example 4. For the experiment in FIG. 10C, the effects of M37 sera on the cross-presentation of M34-T and M34T-MICA cells was determined as in Example 4.

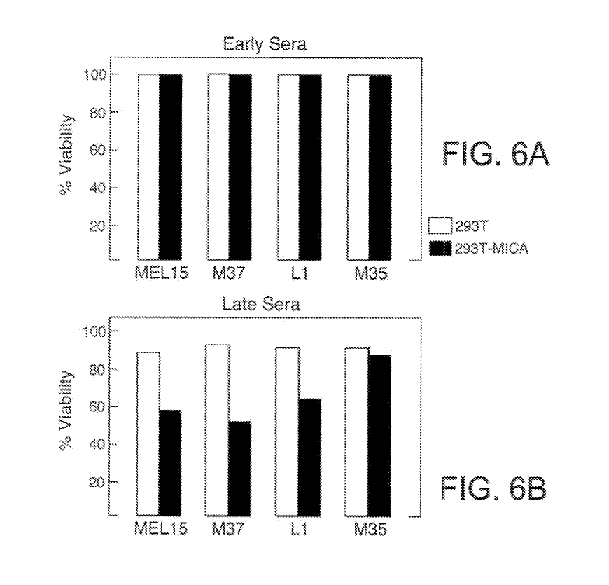

The ability of the immunotherapy-induced anti-MICA antibodies to accomplish tumor lysis through complement fixation was also evaluated. For these studies, 293 embryonic kidney cells were engineered to express high levels of MICA (wild type cells show minimal expression). 293T and 293T-MICA embryonic kidney cells were incubated in patient sera and complement, and cell viability after 6 hours was determined with a 3-(4,5-dimethylthiazol-2-yl)-2,5-diphenyl tetrazolium bromide (MTT) assay. As shown in FIG. 6A, prevaccination sera had no specific effect on complement-mediated lysis. In contrast, the anti-MICA antibodies stimulated by autologous, GM-CSF secreting tumor cell vaccinations or CTLA-4 blockade (“Late sera”) mediated MICA-specific cytotoxicity (FIG. 6B). The differences were abrogated by heat inactivation of the complement. Sera from an immunized melanoma patient without anti-MICA antibodies (M35) failed to induce MICA-dependent lysis.

### Example 6

### Amino Acid Sequence of A Representative Human MICA Polypeptide (Genbank Accession Number NP_{—}000238; GI:4557751)

| | |
|---|---|
| 1 | mglgpvflll agifpfappg aaaephslry nltvlswdgs vqsgfltevh ldgqpflrcd |
| 61 | rqkcrakpqg qwaedvlgnk twdretrdlt gngkdlrmtl ahikdqkegl hslqeirvc |
| 121 | ihednstrss qhfyydgelf lsqnletkew tmpqssraqt lamnvrnflk edamktkthy |
| 181 | hamhadclqe lrrylksgvv lrrtvppmvn vtrseasegn itvtcrasgf ypwnitlswr |
| 241 | qdgvslshdt qqwgdvlpdg ngtyqtwvat ricqgeeqrf tcymehsgnh sthpvpsgkv |
| 301 | lvlqshwqtf hvsavaaaai fviiifyvrc ckkktsaaeg pelvslqvld qhpvgtsdhr |
| 361 | datqlgtqpl msdlgstgst ega |

### Example 7

### Nucleotide Sequence of a Representative Human MICA cDNA (NM_{—}000247; GI:4557750)

| | |
|---|---|
| 1 | cactgcttga gccgctgaga gggtggcgac gtcggggcca tggggctggg cccggtcttc |
| 61 | ctgcttctgg ctggcatctt cccttttgca cctccgggag ctgctgctga gccccacagt |
| 121 | cttcgttata acctcacggt gctgtcctgg gatggatctg tgcagtcagg gtttctcact |
| 161 | gaggtacatc tggatggtca gcccttcctg cgctgtgaca ggcagaaatg cagggcaaag |
| 241 | ccccagggac agtgggcaga agatgtcctg ggaaataaga catgggacag agagaccaga |
| 301 | gacttgacag ggaacggaaa ggacctcagg atgaccctgg ctcatatcaa ggaccagaaa |
| 361 | gaaggcttgc attccctcca ggagattagg gtctgtgaga tccatgaaga caacagcacc |
| 421 | aggagctccc agcatttcta ctacgatggg gagctcttcc tctcccaaaa cctggagact |
| 481 | aaggaatgga caatgcccca gtcctccaga gctcagacct tggccatgaa cgtcaggaat |
| 541 | ttcttgaagg aagatgccat gaagaccaag acacactatc acgctatgca tgcagactgc |
| 601 | ctgcaggaac tacggcgata tctaaaatcc ggcgtagtcc tgaggagaac agtgcccccc |
| 661 | atggtgaatg tcacccgcag cgaggcctca gagggcaaca ttaccgtgac atgcagggct |
| 721 | tctggcttct atccctggaa tatcacactg agctggcgtc aggatggggt atccttgagc |
| 781 | cacgacaccc agcagtgggg ggatgtcctg cctgatggga atggaaccta ccagacctgg |
| 841 | gtggccacca ggatttgcca aggagaggag cagaggttca cctgctacat ggaacacagc |
| 901 | gggaatcaca gcactcaccc tgtgccctct gggaaagtgc tggtgcttca gagtcattgg |
| 961 | cagacattcc atgtttctgc tgttgctgct gctgctattt ttgttattat tattttctat |
| 1021 | gtccgttgtt gtaagaagaa aacatcagct gcagagggtc cagagctcgt gagcctgcag |
| 1081 | gtcctggatc aacacccagt tgggacgagt gaccacaggg atgccacaca gctcggattt |
| 1141 | cagcctctga tgtcagatct tgggtccact ggctccactg agggcgccta gactctacag |
| 1201 | ccaggcagct gggattcaat tccctgcctg gatctcacga gcactttccc tcttggtgcc |
| 1261 | tcagtttcct gacctatgaa acagagaaaa taaaagcact tatttattgt tgttggaggc |
| 1321 | tgcaaaatgt tagtagatat gaggcgttcg cagctgtacc atatt |

### Example 8

### MICA Expression in MGUS and MM

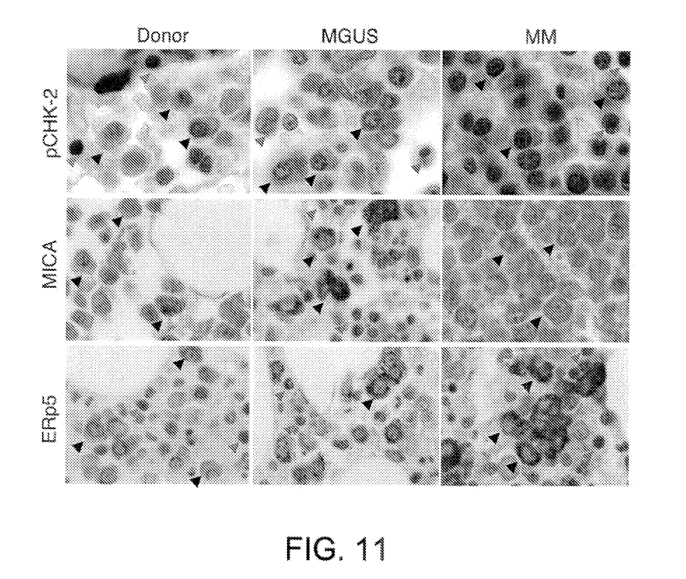

To determine whether the DNA damage response pathway might induce MICA upregulation through the ATM, ATR, chk-1, and chk-2 signaling cascade during plasma cell transformation, we performed immunohistochemistry for phosphorylated chk-2 (thr 68), MICA and ERp5 on bone marrow tissue microarrays. Bone marrow tissue microarrays were prepared from healthy donors (n=10) and patients with untreated MGUS (n=20) or MM (n=40) and stained for phospho-chk-2 (FIG. 1, top row), MICA (FIG. 11, middle row), or ERp5 (FIG. 11, bottom row) according to the method in Example 1. Representative Examples from the Arrays are Shown in FIG. 11 (Magnification: 250×.) The dark arrows indicate plasma cells; the light arrows indicate non-plasma cells. While normal plasma cells showed minimal phospho-chk-2 staining, MGUS and, to a greater degree, MM plasma cells displayed strong reactivity that was primarily nuclear, suggesting that the DNA damage response is evoked early and then sustained during MM progression (FIG. 11, top row). The chk-2 activation in MGUS plasma cells was associated with strong antibody staining for MICA, which was primarily surface, and to a lesser extent cytoplasmic, in contrast to the absence of MICA expression in normal plasma cells (FIG. 11, middle row). Notwithstanding the intense immunoreactivity for phospho-chk-2, MM plasma cells manifested only moderate staining for MICA, which was primarily cytoplasmic. This unexpected expression profile raised the possibility that MM plasma cells might shed surface MICA. Indeed, MM plasma cells evidenced strong antibody staining for ERp5, a protein disulfide isomerase linked to MICA shedding, while MGUS and normal plasma cells were modestly reactive (FIG. 11, bottom row).

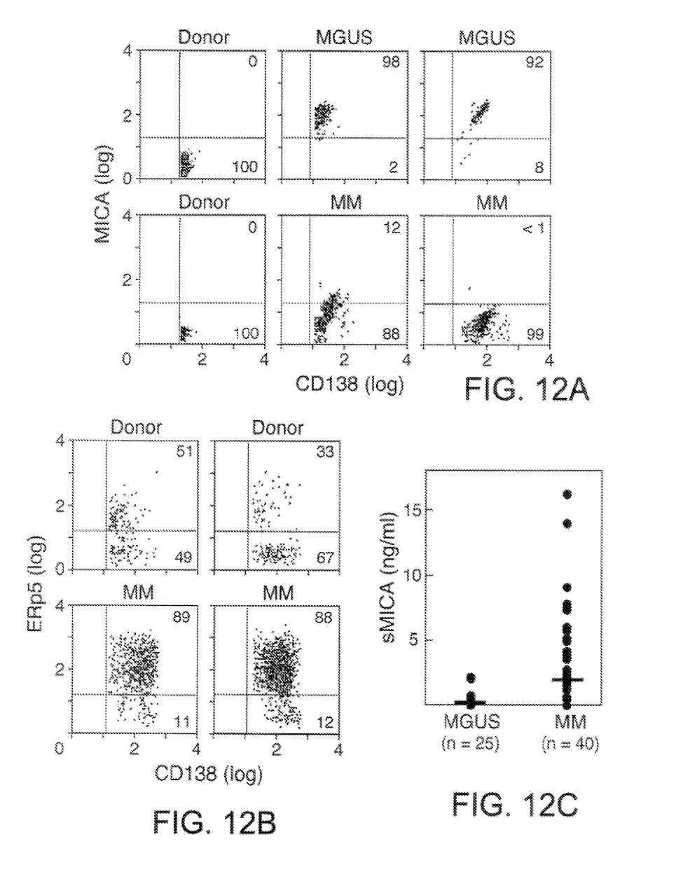

The cell surface expression of MICA and ERp5 was further analyzed by flow cytometry on unfractionated bone marrow samples. Bone marrow aspirates were stained for CD138 and MICA and analyzed with flow cytometry according to the method in Example 1. Consistent with the immunohistochemistry data, CD138+ plasma cells from MGUS patients showed higher surface levels of MICA compared to MM patients, whereas plasma cells from normal donors failed to express MICA (FIG. 12A). Results shown in FIG. 12A are representative of 3 normal donors, 3 MGUS patients, and 7 mM patients studied. Plasma cells from MM patients also displayed much higher levels of ERp5 compared to normal donors (FIG. 12B). Results in FIG. 12B are representative of 3 normal donors and 6 mM patients studied. Sera from MGUS and MM patients were evaluated for sMICA with an ELISA. The upregulation of ERp5 was associated with MICA shedding (FIG. 12C), as MM patients frequently harbored circulating sMICA (median 1.98 ng/ml, range 0 to 16.2; n=40), whereas nearly all MGUS patients (median 0.10, range 0 to 2.18; n=25) and normal donors (not shown) did not (MM versus MGUS patients, p=0.001). Together, these results indicated that the DNA damage response and ERp5 are important determinants of MICA induction and shedding during the progression of MM.

### Example 9

### The Effect of sMICA on Immune Suppression in MM Patients

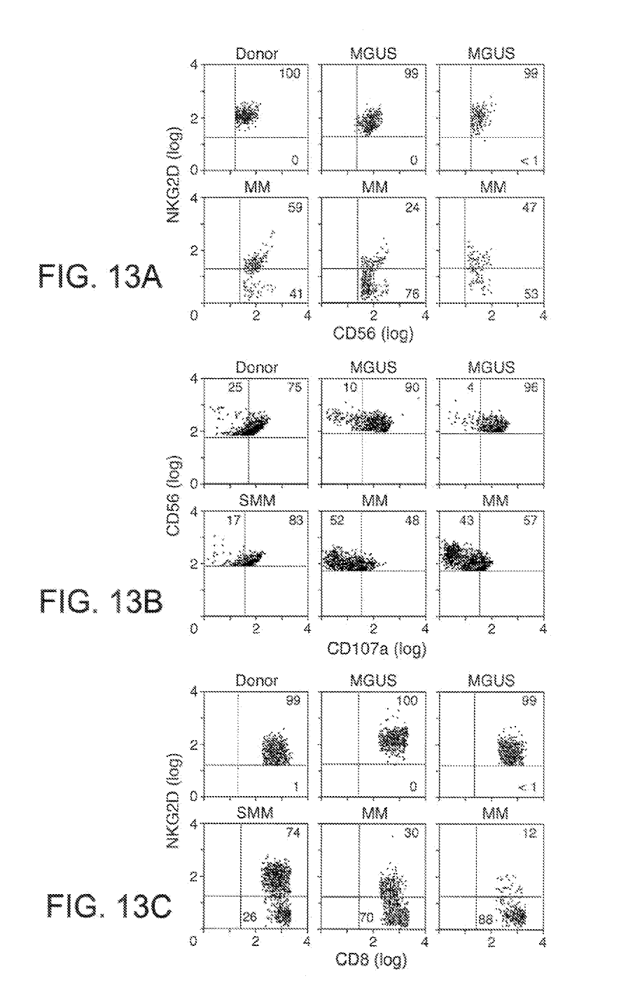

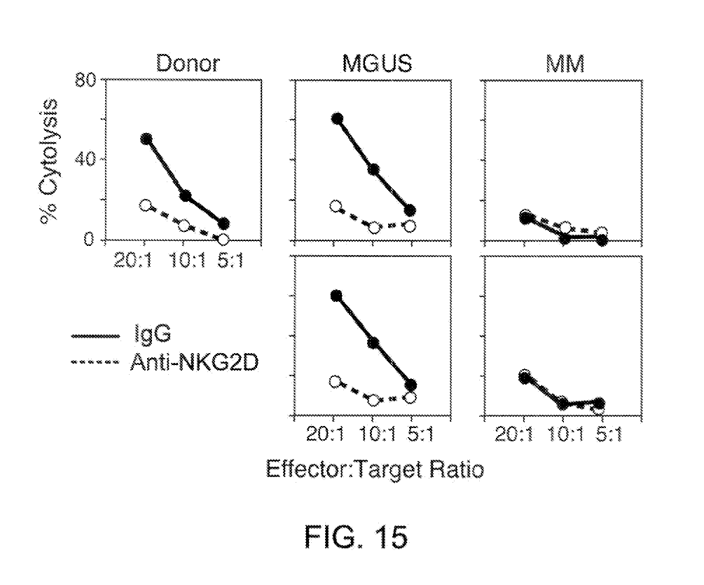

NKG2D expression was analyzed as a function of MM progression. PBMCs from normal donors and patients with MGUS or MM were evaluated for NKG2D expression on gated NK cells (CD56+CD3−) by flow cytometry. Peripheral blood CD56+CD3− NK cells from MGUS patients displayed NKG2D levels that were equivalent to healthy donors, but MM patients with sMICA showed diminished NKG2D (FIG. 13A). Results shown in FIG. 13A are representative of 3 normal donors, 3 MGUS and 7 mM patients studied. To determine the functional consequence of the reduction in NKG2D, we quantified NK cell cytotoxicity towards K562 cells, which is primarily NKG2D dependent, by flow cytometry for CD107a, a lysosomal protein that traffics to the cell surface upon granule exocytosis. First, the NKG2D-dependence of the NK cell cytotoxicity towards K562 cells was confirmed by the experiment shown in FIG. 15. Healthy donor PBMCs were incubated in donor, MGUS, or MM sera for 48 hours and washed; NK cells were then purified with magnetic bead selection and tested for lytic activity against ⁵¹Cr-labeled K562 targets. The NKG2D-dependent lysis was determined with the addition of mAb 1D11 (anti-NKG2D) or isotype control mAb. MM, but not MGUS or donor sera inhibited NKG2D dependent NK cell cytotoxicity. Results are representative of two donors. Points were performed in triplicate; means are shown, with standard deviations less than 10%.

The experiment shown in FIG. 13B quantified NK cell cytotoxicity towards K562 cells. Magnetic bead-purified healthy donor, MGUS, or MM patient NK cells were tested for lytic activity against K562 targets by surface CD107a mobilization. The lytic activity of NK cells from MGUS patients was comparable or increased compared to normal donors, while MM patients with sMICA manifested decreased CD107a mobilization (FIG. 13B). SMM is a smouldering MM patient. Results shown in FIG. 13B are representative of 3 normal donors, 3 MGUS, 3 mM, and 1 SMM patient studied. The importance of sMICA was underscored by the minimal impairment of cytotoxicity in NK cells from a patient with smouldering multiple myeloma (SMM), who did not harbor shed ligand and maintained intact NKG2D. Peripheral blood CD3+CD8+ cytotoxic T cells from MM patients with sMICA also evidenced diminished NKG2D, in contrast to the normal levels in MGUS subjects and the modest decrease in the SMM patient (FIG. 13C). Results shown in FIG. 13C are representative of 3 normal donors, 3 MGUS, 7 mM, and 1 SMM patient studied. These findings suggest that sMICA contributes to the suppression of innate and adaptive immunity during the progression of MM.

### Example 10

### MGUS Patients Generate Anti-MICA Antibodies

The production of anti-MICA antibodies during the pathogenesis of MM was analyzed using sera from MGUS, MM patients and healthy donors. Sera were diluted 1:100 and analyzed by ELISA with recombinant MICA protein. Reactivity was determined with a pan-IgG secondary. As shown in FIG. 4A, MGUS patients (n=25) frequently developed anti-MICA antibodies (median absorbance at 1:100 sera dilution, 0.156, with a range from 0.054 to 0.715), but MM patients (n=40) displayed only weak reactivity (median 0.067, range 0.038 to 0.13), which was minimally increased above normal donors (n=14) (median 0.049, range 0.024 to 0.092). The differences between the MGUS patients and MM patients were highly significant (p=0.0002).

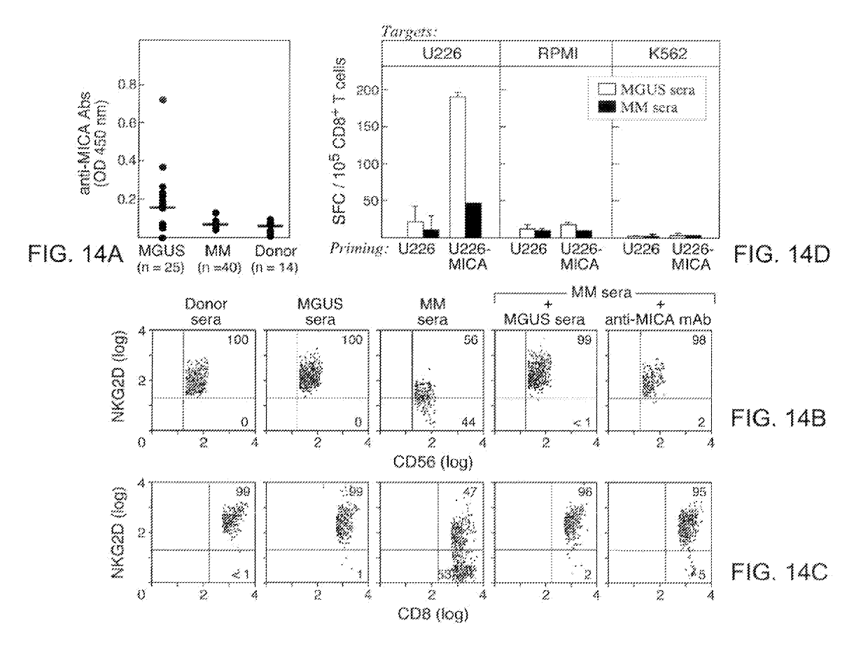

To characterize the biologic properties of the anti-MICA antibodies, we tested their ability to antagonize the downregulation of NKG2D evoked by sMICA. Peripheral blood CD56+CD3− NK cells were purified from healthy donors, incubated in various sera for 48 hours, then NKG2D expression on gated NK cells (CD56+, CD3−) was determined with flow cytometry. Anti-MICA monoclonal antibodies were added where indicated. As shown in FIG. 14B, MM, but not MGUS or donor sera resulted in diminished NKG2D levels (FIG. 14B) and impaired killing of K562 targets (FIG. 15). However, as shown in FIG. 14C, the admixing of MGUS and MM sera (1:1 ratio) blocked the decrease in NKG2D expression at comparable efficiency to the addition of anti-MICA monoclonal antibodies. Gated CD3+CD8+ T cells are shown. Similar results were observed using three different MGUS and MM sera samples. MGUS sera similarly prevented the reduction in NKG2D on purified donor CD8+ peripheral blood T cells exposed to MM sera (FIG. 14C). These results indicate that the anti-MICA antibodies generated in MGUS are neutralizing, and that sMICA, but not other factors such as TGF-β, underlies the decreased NKG2D levels in MM.

To determine whether the anti-MICA antibodies also recognized cell surface ligand, we evaluated the cross-presentation of U226 mM cells. While this line did not express MICA at baseline, gamma-irradiation augmented expression. To optimize the analysis, however, we used retroviral mediated gene transfer to engineer U226 cells with stable, high-level MICA expression (U226-MICA). Dendritic cells were generated from a healthy donor by culture of adherent donor PBMCs in GM-CSF and IL-4, loaded with MGUS or MM sera-coated U226 or U226-MICA MM cells, matured with LPS, and used to stimulate autologous purified CD8+ T cells for seven days. IFN-γ production was measured by ELISPOT against K562 cells or dendritic cells loaded with U226 or RPMI-8226 mM cells. No reactivity against unpulsed dendritic cells or dendritic cells loaded with un-opsonized tumors was observed. As shown in FIG. 14D, MGUS sera mediated efficient MICA dependent cross-presentation of U226 cells, resulting in tumor specific CD8+ T cells, but little reactivity to RPMI MM cells or K562 targets (FIG. 14D). In contrast, MM sera were minimally active. These findings indicate that anti-MICA antibodies in MGUS sera effectively opsonize malignant plasma cells, which might contribute to the induction and/or maintenance of potent anti-tumor cytotoxic CD8+ T cells during the early stages of MM.

### Example 11

### Bortezomib Increased MICA Expression in Some MM Cells

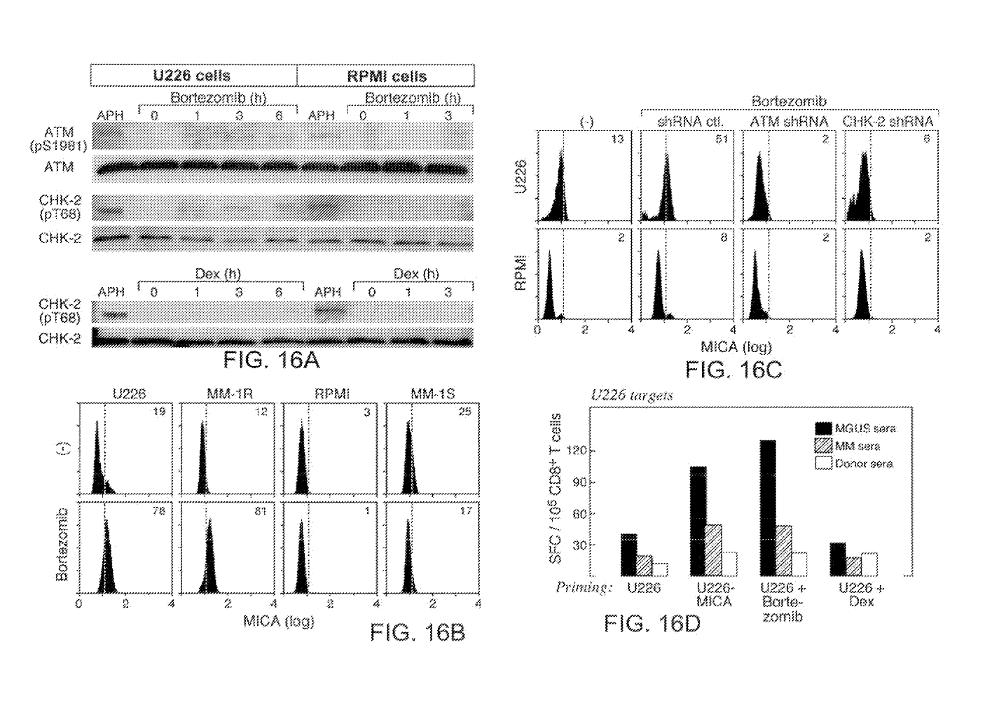

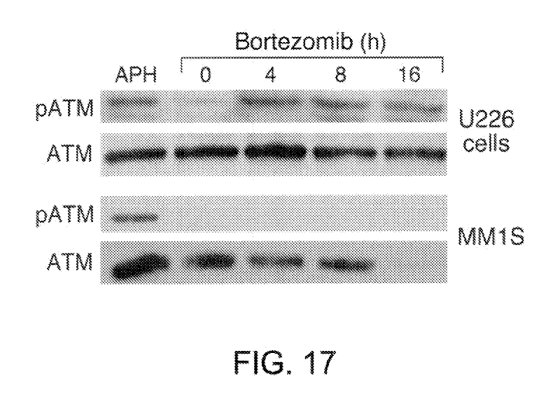

The effect of the proteasome inhibitor Bortezomib, a MM therapeutic, on MICA expression and the DNA damage response pathway was analyzed. U226 and RPMI-8226 MM cells were treated with 20 μM Bortezomib, 250 μg/ml dexamethasone or 10 μg/ml aphidicolin (a DNA polymerase inhibitor that served as a control for inducing DNA damage) for the indicated times. Cell lysates were prepared and evaluated for phospho-ATM (ser 1981), ATM, phospho-chk-2 (thr 68) or chk-2 by immunoblotting. As shown in FIG. 16A, Bortezomib triggered the rapid phosphorylation of ATM (ser 1981) and chk-2 (thr 68) in U226 mM, but not RPMI MM cells, whereas dexamethasone, another agent with anti-MM clinical efficacy, failed to activate the pathway in either line. In another MM cell line, MM-1S, Bortezomib stimulated the degradation of ATM. MM cells were exposed to 10 μM Bortezomib for the indicated times and then lysed. Immunoblotting was performed for anti-phospho-ATM (ser 1981) and total ATM. 10 μg/ml aphidicolin, a DNA polymerase inhibitor, was used as a positive control for activation of the DNA damage response. As shown in FIG. 17, Bortezomib induced ATM phosphorylation in U226 cells, but triggered the degradation of ATM in MM-1S cells.

Consistent with the phosphorylation of ATM and chk-2 in U226 mM and MM-1R MM cells, Bortezomib increased the surface expression of MICA in these lines, but not in RPMI or MM-1S cells (FIG. 16B). For the experiment shown in FIG. 16B, the indicated MM cells were treated with 20 μM Bortezomib overnight and surface MICA expression was determined with flow cytometry. In contrast, dexamethasone did not alter MICA levels in any of the lines tested. The Bortezomib mediated upregulation of MICA in U226 mM cells required the DNA damage response, because shRNA silencing of ATM or chk-2 blocked ligand induction (FIG. 16C). For the experiment shown in FIG. 16C, U226 and RPMI-8226 mM cells were transfected with plasmids encoding control, ATM or chk-2 shRNAs, exposed to Bortezomib overnight, and assayed for MICA expression with flow cytometry. Bortezomib also enhanced the immunogenicity of U226 MM cells, as MGUS sera effectuated the dendritic cell cross-presentation of Bortezomib treated cells as efficiently as U226-MICA cells, thereby engendering potent anti-MM CD8+ T cell responses (FIG. 16D). For the experiment shown in FIG. 16D, dendritic cells were generated from adherent donor PBMCs, loaded with MGUS, MM, or normal sera-coated U226, U226-MICA, Bortezomib treated U226, or dexamethasone treated U226 MM cells, matured with LPS, and used to stimulate autologous purified CD8+ T cells for seven days. IFN-γ production was measured by ELISPOT against dendritic cells loaded with U226 MM cells. No reactivity against K562 cells, unpulsed dendritic cells or dendritic cells loaded with un-opsonized tumors was observed. Results are representative of four donors. Points were performed in triplicate; means are shown, with standard deviations less than 10%. Other studies showed that shRNA silencing of ATM or chk-2 abrogated the immunostimulatory effects of Bortezomib in U226 cells, whereas the cross-presentation of dexamethasone treated cells by MGUS sera was equivalent to untreated U226 cells, highlighting the specificity for MICA. These findings suggest that anti-MICA monoclonal antibodies may potentiate the clinical activity of Bortezomib in some patients.

### Example 12

### GM-CSF Secreting RENCA Cell Vaccines Stimulated a Broad Humoral Response

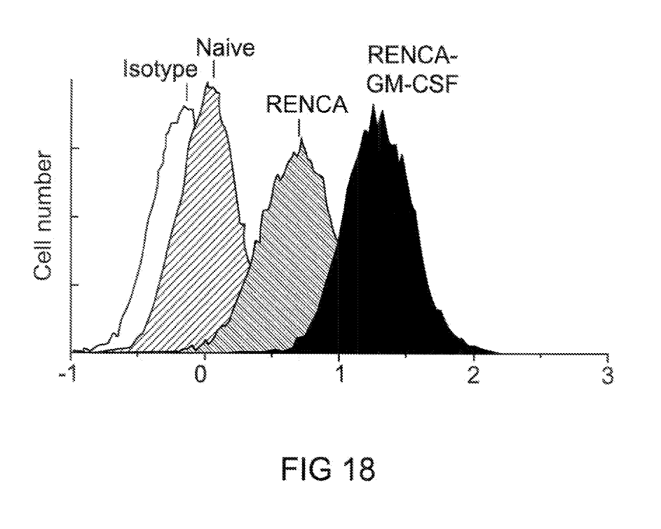

Sera were collected from syngeneic Balb/c mice that were either naïve or immunized ten times at weekly intervals with irradiated GM-CSF secreting or parental RENCA cells. Sera were diluted 1:100 and evaluated for reactivity against live RENCA cells by flow cytometry. An anti-mouse pan-IgG secondary antibody was used to detect isotypes that depend upon CD4+ T cell help for class switching. Both types of RENCA vaccines administered at weekly intervals evoked antibody responses that increased steadily with repetitive immunizations, but, as shown in FIG. 18, irradiated, GM-CSF secreting RENCA cells stimulated stronger reactivity than irradiated wild type cells, while naïve mice displayed minimal staining.

To identify the targets of these humoral responses, we constructed a cDNA expression library in bacteriophage using mRNA isolated from wild type RENCA cells. Sera collected from mice immunized ten times with irradiated, wild type or GM-CSF secreting RENCA cells were diluted 1:300 and employed for the library screening. Immunoreactive plaques were detected with an anti-mouse IgG secondary antibody and purified through further re-platings. The screening with sera from mice vaccinated with wild type RENCA cells yielded only 3 clones that represented 2 distinct gene products, but GM-CSF stimulated the detection of 177 clones, which represented 26 distinct gene products. Additional phage plate assays revealed that sera from mice immunized with GM-CSF secreting cells also recognized the 2 antigens identified with parental RENCA vaccines, while sera from mice immunized with wild type cells failed to detect any of the GM-CSF associated targets. Moreover, sera from naïve mice did not react with any of the 28 antigens characterized in the two screens. Together, these findings indicate that although vaccination with irradiated wild type cells modestly enhanced humoral recognition of RENCA cells, GM-CSF secretion resulted in a marked diversification of the antigenic repertoire targeted by antibodies.

### Example 12

### RENCA Vaccine Targets are Involved in Oncogenic Pathways

Of the 28 distinct antigens recognized by sera from mice immunized with GM-CSF producing cells, 21 encoded known proteins, many of which have been previously implicated in transformation, as shown in Table 2. These gene products could be grouped based upon putative roles in cell function. One set of antigens contributes to tumor cell migration. Among these, CD44, which is also aberrantly expressed in human renal cell carcinomas, mediates tumor cell adhesion to endothelial cells and the extra-cellular matrix, while rho-associated coiled-coil forming kinase-2 (ROCK2) modifies the cellular cytoskeleton to promote metastasis. A second group of targets, which included eukaryotic translational initiation factor 4A1 (eIF4A-1), ribosomal protein L15 (RPL15), and the proteasome subunit, beta type 5 (PSMB5), are involved in protein translation and degradation, two pathways that are critical to the regulation of tumor cell growth and survival. A third set of antigens consisted of heat-responsive protein 12 (HRP12), autophagy related 3 (atg3), and autophagy related 12 (atg12), which enhance the resistance of tumor cells to various forms of stress. A fourth group of targets participates in intermediary metabolism and includes farnesyl diphosphate synthetase (FDPS), a key enzyme in the mevalonate biosynthetic pathway that generates cholesterol and isoprenoids, and ATP synthase mitochondrial F1 complex delta subunit (atp5d), an important component of oxidative phosphorylation. A final set of antigens encompassed nuclear proteins that contribute to chromatin, DNA repair, and transcriptional control.

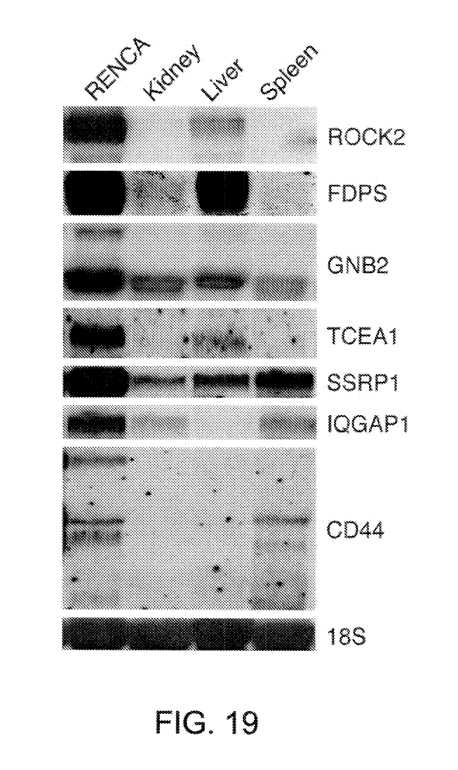

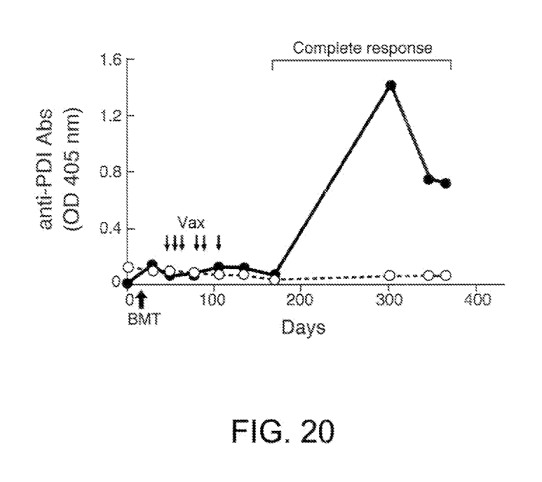

The sequencing of the 21 known vaccine targets failed to reveal any alterations compared to the GenBank database, eliminating mutation as a possible mechanism for their immunogenicity. Nonetheless, previous work suggested that the increased expression of normal differentiation proteins, such as Her2/neu and MART-1, might provoke immune recognition of tumor cells. Since transformation may involve the aberrant activation of specific oncogenic pathways, we thus characterized the expression of selected RENCA antigens by northern analysis. RNA was isolated from RENCA cells and normal kidney, liver, and spleen, and the transcript levels for multiple RENCA antigens were determined by northern analysis. The 18S ribosomal RNA served as a loading control. As shown in FIG. 19, all of the gene products examined, including ROCK2, FDPS, guanine nucleotide-binding protein P2 subunit (GNB2), transcription elongation factor A (S11) 1 (TCEA1), structure-specific recognition protein 1 (SSRP1), IQGAP1, and CD44, displayed increased transcript levels in RENCA cells compared to normal kidney, liver, and spleen (with the exception of FDPS that was expressed to a comparable degree in the liver, the major site of cholesterol biosynthesis). High levels of SSRP1 and surface CD44 protein were also confirmed with western analysis and flow cytometry of RENCA cells respectively. Together, these results indicated that the humoral response stimulated by vaccination with GM-CSF secreting RENCA cells targeted gene products that are over-expressed and involved in diverse oncogenic pathways.

TABLE 2
RENCA antigens targeted by vaccine-induced antibodies.

| ANTIGEN | SYMBOL | GENE ID | FUNCTION |
|---|---|---|---|
| CD44 | CD44 | 12505 | Migration/invasion |
| Guanine nucleotide-binding protein, □2 subunit | GNB2 | 14693 | |
| Rho-associated coiled-coil forming kinase 2 | ROCK2 | 19878 | |
| IQ motif GTPase activating protein 1 | IQGAP1 | 29875 | |
| Eukaryotic translation initiation factor 4A1 | eIF4A-1 | 13681 | Protein homeostasis |
| Ribosomal protein L15 | RPL15 | 66480 | |
| Protein disulfide isomerase | PDI | 18453 | |
| ADP-ribosylation factor 4 | ARF4 | 11843 | |
| Proteasome subunit, beta type 5 | PSMB5 | 19173 | |
| Heat-responsive protein 12 | HRP12 | 15473 | Stress responses |
| Autophagy related 3 | Atg3 | 67841 | |
| Autophagy related 12 | Atg12 | 67526 | |
| Farnesyl diphosphate synthetase | FDPS | 110196 | Metabolism |
| Acetyl-Coenzyme A acetytransferase 2 | ACAT2 | 110460 | |
| Aldose reductase | Akr1b3 | 11677 | |
| ATP synthase, mitochondrial F1 complexdelta subuni [symbol] | Atp5d | 66043 | |
| Pre-B-cell colony-enhancing factor 1 | PBEF1 | 59027 | |
| H1 histone family, member 0 | H1(0) | 14958 | Chromatin/transcription |
| Structure-specific recognition protein 1 | SSRP1 | 20833 | |
| Transcription elongation factor A (SII) 1 | TCEA1 | 21399 | |
| Heterogeneous nuclear ribonucleoprotein C | hnRNPC1 | 15381 | |

[symbol]

indicates data missing or illegible when filed

### Example 13

### Protein Disulfide Isomerase is Immunogenic in Myeloid Leukemia

The human orthologs of ROCK2, SSRP1, eIF4A, IQGAP, aldose reductase (Akr1b3), acetyl-coenzyme A acetylransferase 2 (ACAT2), and heterogeneous nuclear ribonucleoprotein C (hnRNPC1) were previously deposited in the Cancer Immunome Database (www2.licr.org/cancerimmunomeDB), reflecting their earlier identification as antibody targets in cancer patients through the screening of human tumor-derived cDNA expression libraries. This conservation of immunogenicity supports is consistent with the idea that the characterization of vaccine responses in a murine model might prove informative for the analysis of human anti-tumor immunity. The most frequently recognized antigen in the RENCA library screening was protein disulfide isomerase (PDI), which accounted for 86% of the clones detected with sera from mice immunized with GM-CSF secreting cells.

To examine whether PDI was immunogenic in cancer patients, we established an ELISA with recombinant full-length human protein and evaluated sera from 46 metastatic melanoma, 22 metastatic non-small cell lung carcinoma, 2 metastatic ovarian carcinoma, and 12 acute myeloid leukemia patients who were enrolled on Phase I clinical trials of vaccination with irradiated, autologous tumor cells engineered to secrete GM-CSF. We used a pan-human IgG secondary antibody to measure preferentially those isotypes dependent upon CD4+ T cell help for class switching. Humoral responses to PDI proved uncommon in this cohort, as only one melanoma and one leukemia patient were reactive, whereas 30 healthy donors were negative. Although the anti-PDI antibodies in the melanoma patient were detectable prior to vaccination and not altered with therapy, a longitudinal analysis of the leukemia subject uncovered an intriguing correlation between humoral immunity and clinical outcome. As shown in FIG. 3, a longitudinal analysis of humoral reactivity to human PDI (closed circles) and human PBEF (open circles) was performed with serial sera samples diluted 1:100 and a secondary anti-human pan-IgG secondary antibody. This leukemia patient first underwent a non-myeloablative allogeneic bone marrow transplant (BMT) and then was vaccinated with irradiated, autogous GM-CSF secreting leukemia cells as indicated. Reduction of the immunosuppression for prophylaxis of graft-versus-host disease preceded the complete hematologic response, which has been maintained with 17 months of follow-up.

This subject with refractory acute myeloid leukemia underwent a non-myeloablative allogeneic bone marrow transplant and then received irradiated, autologous GM-CSF secreting tumor cells early post-transplant. After the completion of vaccination in the human protocol, the immunosuppressive therapy for prophylaxis against graft-versus-host disease was reduced, and the leukemia patient achieved a complete hematologic response that was temporally associated with the development of high titer anti-PDI antibodies. No humoral reactivity against human pre-B-cell colony enhancing factor (PBEF), an ortholog of another RENCA antigen identified in the murine screen (Table 2), was observed, though, establishing the specificity of the antibody response.

### Example 14

### Humoral Responses to Protein Disulfide Isomerase ERp5

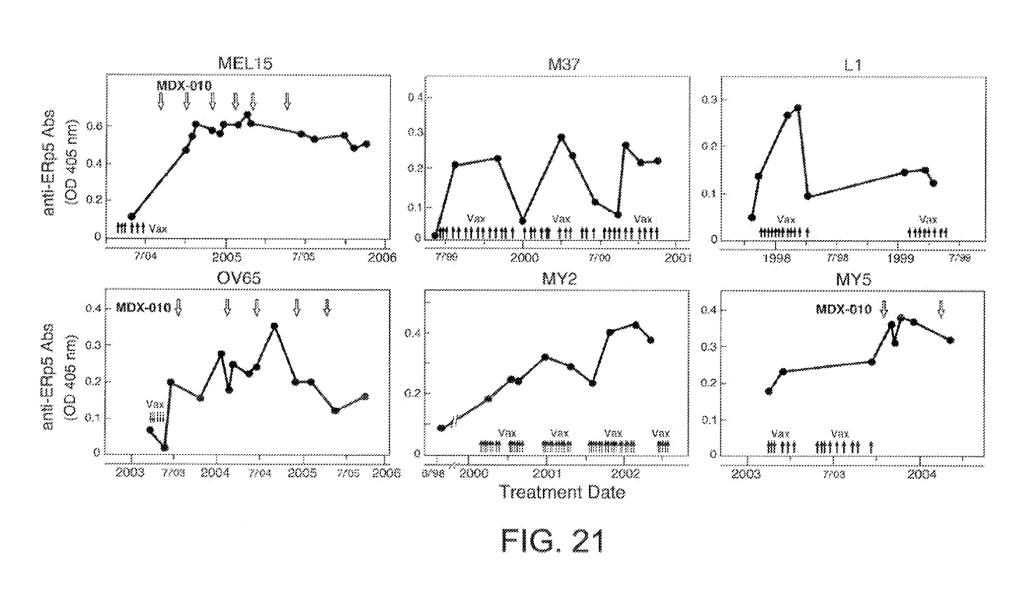

The immunogenicity of the protein disulfide isomerase ERp5 was explored. We established an ELISA with recombinant human ERp5 protein and evaluated the development of antigen-specific IgG antibodies in the same patient cohort used for the PDI analysis. A longitudinal analysis, shown in FIG. 21, of antibodies to human ERp5 was performed with serial sera samples diluted 1:50 and a secondary anti-human pan-IgG antibody. MEL15 and M37 are metastatic melanoma patients, L1 is a metastatic non-small cell lung carcinoma patient, OV65 is a metastatic ovarian carcinoma patient, and MY2 and MY5 are acute myeloid leukemia patients. Upward arrows denote vaccination with irradiated, autologous, GM-CSF secreting tumor cells. Downward arrows indicate the infusion of a fully human anti-CTLA-4 blocking monoclonal antibody (MDX-010; Ipilumimiab®). Remarkably, three of the four subjects previously shown to mount anti-MICA antibodies as a function of immunotherapy also generated humoral responses to ERp5 (FIG. 21, top row). Metastatic melanoma patient MEL15 achieved an ongoing near complete response to vaccination and CTLA-4 blockade (36+ mos); metastatic melanoma subject M37 remains in a complete response eight years following vaccination; and metastatic non-small cell lung carcinoma patient L1 achieved stable disease of two years duration after immunization. Furthermore, we identified three additional subjects without anti-MICA antibodies who similarly developed humoral reactions to ERp5 as a consequence of GM-CSF secreting vaccines and/or CTLA-4 antibody blockade (FIG. 21, bottom row). These included an advanced ovarian carcinoma patient who achieved an ongoing partial response (3.5+ years) and 2 advanced myeloid leukemia subjects who demonstrated prolonged stable disease (30 and 18 months); these leukemia patients were enrolled in a vaccination protocol that did not involve allogeneic bone marrow transplant (this trial will be reported in detail elsewhere). Together, these investigations delineate a striking correlation between humoral reactions to ERp5 and immune-mediated tumor destruction that is operative in four different types of human malignancies.

### Example 15

### Amino Acid Sequence of a Representative Human PDI Polypeptide (Genbank Accession Number EAW89696; gi:119610102)

| | |
|---|---|
| 1 | metrlpprni qdvesdsakq flqaaeaidd ipfgitsnsd vfskyqldkd gvvlfkkfde |
| 61 | grnnfegevt kenlldfikh nqlplvieft eqtapkifgg eikthillfl pksvsdydgk |
| 121 | lsnfktaaes fkgkilfifi dsdhtdnqri leffglkkee cpavrlitle eemtkykpes |
| 181 | eeltaerite fchrflegki kphlmsqelp edwdkqpvkv lvgknfedva fdekknvfve |
| 241 | fyapwcghck qlapiwdklg etykdheniv iakmdstane veavkvhsfp tlkffpasad |
| 301 | rtvidynger tldgfkkfle sggqdyagdd ddledleeae epdmeedddq kavkdel |

### Example 16

### Nucleotide Sequence Encoding a Representative Human PDI Polypeptide (Genbank Accession Number NM_{—}000918; gi:20070124)

| | |
|---|---|
| 1 | cccggcggcg ccaaccgaag cgccccgcct gatccgtgtc cgacatgctg cgccgcgctc |
| 61 | tgctgtgcct ggccgtggcc gccctggtgc gcgccgacgc ccccgaggag gaggaccacg |
| 121 | tcctggtgct gcggaaaagc aacttcgcgg aggcgctggc ggcccacaag tacctgctgg |
| 181 | tggagttcta tgccccttgg tgtggccact gcaaggctct ggcccctgag tatgccaaag |
| 241 | ccgctgggaa gctgaaggca gaaggttccg agatcaggtt ggccaaggtg gacgccacgg |
| 301 | aggagtctga cctggcccag cagtacggcg tgcgcggcta tcccaccatc aagttcttca |
| 361 | ggaatggaga cacggcttcc cccaaggaat atacagctgg cagagaggct gatgacatcg |
| 421 | tgaactggct gaagaagcgc acgggcccgg ctgccaccac cctgcctgac ggcgcagctg |
| 481 | cagagtcctt ggtggagtcc agcgaggtgg ctgtcatcgg cttcttcaag gacgtggagt |
| 541 | cggactctgc caagcagttt ttgcaggcag cagaggccat cgatgacata ccatttggga |
| 601 | tcacttccaa cagtgacgtg ttctccaaat accagctcga caaagatggg gttgtcctct |
| 661 | ttaagaagtt tgatgaaggc cggaacaact ttgaagggga ggtcaccaag gagaacctgc |
| 721 | tggactttat caaacacaac cagctgcccc ttgtcatcga gttcaccgag cagacagccc |
| 781 | cgaagatttt tggaggtgaa atcaagactc acatcctgct gttcttgccc aagagtgtgt |
| 841 | ctgactatga cggcaaactg agcaacttca aaacagcagc cgagagcttc aagggcaaga |
| 901 | tcctgttcat cttcatcgac agcgaccaca ccgacaacca gcgcatcctc gagttctttg |
| 961 | gcctgaagaa ggaagagtgc ccggccgtgc gcctcatcac cctggaggag gagatgacca |
| 1021 | agtacaagcc cgaatcggag gagctgacgg cagagaggat cacagagttc tgccaccgct |
| 1081 | tcctggaggg caaaatcaag ccccacctga tgagccagga gctgccggag gactgggaca |
| 1141 | agcagcctgt caaggtgctt gttgggaaga actttgaaga cgtggctttt gatgagaaaa |
| 1201 | aaaacgtctt tgtggagttc tatgccccat ggtgtggtca ctgcaaacag ttggctccca |
| 1261 | tttgggataa actgggagag acgtacaagg accatgagaa catcgtcatc gccaagatgg |
| 1321 | actcgactgc caacgaggtg gaggccgtca aagtgcacag cttccccaca ctcaagttct |
| 1381 | ttcctgccag tgccgacagg acggtcattg attacaacgg ggaacgcacg ctggatggtt |
| 1441 | ttaagaaatt cctggagagc ggtggccagg atggggcagg ggatgatgac gatctcgagg |
| 1501 | acctggaaga agcagaggag ccagacatgg aggaagacga tgatcagaaa gctgtgaaag |
| 1561 | atgaactgta atacgcaaag ccagacccgg gcgctgccga gacccctcgg gggctgcaca |
| 1621 | cccagcagca gcgcacgcct ccgaagcctg cggcctcgct tgaaggaggg cgtcgccgga |
| 1681 | aacccaggga acctctctga agtgacacct cacccctaca caccgtccgt tcacccccgt |
| 1741 | ctcttccttc tgcttttcgg tttttggaaa gggatccatc tccaggcagc ccaccctggt |
| 1801 | ggggcttgtt tcctgaaacc atgatgtact ttttcataca tgagtctgtc cagagtgctt |
| 1861 | gctaccgtgt tcggagtctc gctgcctccc tcccgcggga ggtttctcct ctttttgaaa |
| 1921 | attccgtctg tgggattttt agacattttt cgacatcagg gtatttgttc caccttggcc |
| 1981 | aggcctcctc ggagaagctt gtcccccgtg tgggagggac ggagccggac tggacatggt |
| 2041 | cactcagtac cgcctgcagt gtcgccatga ctgatcatgg ctcttgcatt tttgggtaaa |
| 2101 | tggagacttc cggatcctgt cagggtgtcc cccatgcctg gaagaggagc tggtggctgc |
| 2161 | cagccctggg gcccggcaca ggcctgggcc ttccccttcc ctcaagccag ggctcctcct |
| 2221 | cctgtcgtgg gctcattgtg accactggcc tctctacagc acggcctgtg gcctgttcaa |
| 2281 | ggcagaacca cgacccttga ctcccgggtg gggaggtggc caaggatgct ggagctgaat |
| 2341 | cagacgctga cagttcttca ggcatttcta tttcacaatc gaattgaaca cattggccaa |
| 2401 | ataaagttga aattttacca ccaaaaaaaa aaaaaaaa |

### Example 17

### Amino Acid Sequence of a Representative Human ERp5 Polypeptide (Genbank accession Number AAR01312; gi:1265493)

| | |
|---|---|
| 1 | mallvlglvs ctfflavngl ysssddviel tpsnfnrevi qsdslwlvef yapwcghcqr |
| 61 | ltpewkkaat alkdvvkvga vdadkhhslg gqyyvqgfpt ikifgsnknr pedyqggrtg |
| 121 | eaivdaalsa lrqlvkdrlg grsggyssgk qgrsdssskk dvieltddsf dknvldsedv |
| 181 | wmvefyapwc ghcknlepew aaaasevkeq tkgkvklaav datvnqvlas rygirgfpti |
| 241 | kifqkgespv dydggrtrsd ivsraldlfs dnapppelle iinediakrt ceehqlcvva |
| 301 | vlphildtga agrnsylevl lkladkykkk mwgwlwteag aqseletalg iggtgypama |
| 361 | ainarkmkfa llkgsfseqg ineflrelsf grgetapvgg gafptivere pwdgrdgelp |
| 421 | veddidlsdv elddlgkdel |

### Example 18

### Nucleotide sequence encoding a representative human ERp5 polypeptide (Genbank accession number BC001312; gi:12654930)

| | |
|---|---|
| 1 | ggcctggggc gggacgtggg cgcgggggcg cggcgtgcgg cacgctgcag ggctgaagcg |
| 61 | gcggcggcgg tggggactgc acgtagcccg gcgctcggca tggctctcct ggtgctcggt |
| 121 | ctggtgagct gtaccttctt tctggcagtg aatggtctgt attcctctag tgatgatgtg |
| 181 | atcgaattaa ctccatcgaa tttcaaccga gaagttattc agagtgatag tttgtggctt |
| 241 | gtagaattct atgctccatg gtgtggtcac tgtcaaagat taacaccaga atggaagaaa |
| 301 | gcagcaactg cattaaaaga tgttgtcaaa gttggtgcag ttgatgcaga taagcatcat |
| 361 | tccctaggag gtcagtatgg tgttcaggga tttcctacca ttaagatttt tggatccaac |
| 421 | aaaaacagac cagaagatta ccaaggtggc agaactggtg aagccattgt agatgctgcg |
| 481 | ctgagtgctc tgcgccagct cgtgaaggat cgcctcgggg gacggagcgg aggatacagt |
| 541 | tctggaaaac aaggcagaag tgatagttca agtaagaagg atgtgattga gctgacagac |
| 601 | gacagctttg ataagaatgt tctggacagt gaagatgttt ggatggttga gttctatgct |
| 661 | ccttggtgtg gacactgcaa aaacctagag ccagagtggg ctgccgcagc ttcagaagta |
| 721 | aaagagcaga cgaaaggaaa agtgaaactg gcagctgtgg atgctacagt caatcaggtt |
| 781 | ctggcctccc gatacgggat tagaggattt cctacaatca agatatttca gaaaggcgag |
| 841 | tctcctgtgg attatgacgg tgggcggaca agatccgaca tcgtgtcccg ggcccttgat |
| 901 | ttgttttctg ataacgcccc acctcctgag ctgcttgaga ttatcaacga ggacattgcc |
| 961 | aagaggacgt gtgaggagca ccagctctgt gttgtggctg tgctgcccca tatccttgat |
| 1021 | actggagctg caggcagaaa ttcttatctg gaagttcttc tgaagttggc agacaaatac |
| 1081 | aaaaagaaaa tgtgggggtg gctgtggaca gaagctggag cccagtctga acttgagacc |
| 1141 | gcgttgggga ttggagggtt tgggtacccc gccatggccg ccatcaatgc acgcaagatg |
| 1201 | aaatttgctc tgctaaaagg ctccttcagt gagcaaggca tcaacgagtt tctcagggag |
| 1261 | ctctcccttg ggcgtggctc cacggcacct gtaggaggcg gggctttccc taccatcgtt |
| 1321 | gagagagagc cttgggacgg cagggatggc gagcttcccg tggaggatga cattgacctc |
| 1381 | agtgatgtgg agcttgatga cttagggaaa gatgagttgt gagagccaca acagaggctt |
| 1441 | cagaccattt tcttttcttg ggagccagtg gatttttcca gcagtgaagg gacattctct |
| 1501 | acactcagat gactctacca gtggcctttt aaccaagaag tagtacttga ttggtcattt |
| 1561 | gaaaacactg caacagtgaa cttttgcatc tcaagaaaac attgaaaaat tctatgaatt |
| 1621 | gttgtagccg gtgaattgag tcgtattctg tcacataaca ttttgaagaa aacttggctg |
| 1681 | tcgaaacatt tttctctctg actgctgctt gaatgttctt ggaggctgtt tcttatgtat |
| 1741 | gggttttttt taatgtgatc ccttcatttg aatattaatg gctttttcca ttaaagaata |
| 1801 | aaatattttg gacaatgcca aaaaaaaaaa aaaaaaaaaa aaaaaaa |

A number of embodiments of the invention have been described. Nevertheless, it will be understood that various modifications may be made without departing from the spirit and scope of the invention. Accordingly, other embodiments are within the scope of the following claims.

1. A method of treating a cancer, or a symptom of cancer or cancer progression in a subject, the method comprising administering to the subject an effective amount of a MICA modulating composition.

2. The method of claim 1, wherein the MICA modulating composition comprises an anti-MICA antibody, an anti-protein disulfide isomerase (PDI) antibody or a combination thereof.

3. (canceled)

4. The method of claim 1, wherein the MICA modulating composition comprises an agent that modulates MICA shedding.

5. (canceled)

6. (canceled)

7. The method of claim 1 further comprising administering one or more tumor cell antigens that elicit an immune response against a tumor.

8. The method of claim 7, wherein the tumor cell antigens include autologous tumor cells.

9. (canceled)

10. The method of claim 1 further comprising administering an anti-CTLA-4 antibody.

11-13. (canceled)

14. The method of claim 1, wherein the cancer expresses elevated levels or activity of MICA.

15-27. (canceled)

28. The method of claim 2, wherein the anti-MICA antibody reduces the level of soluble MICA (sMICA) in the subject.

29. (canceled)

30. A method of eliciting an immune response against a cancer in a subject having a MICA-expressing cancer, comprising: a) identifying a patient having a MICA-expressing cancer; and b) administering an effective amount of an anti-MICA antibody.

31. (canceled)

32. The method of claim 30, wherein the immune response is selected from the group consisting of at least one of increased NKG2D-dependent cell killing, increased anti-tumor CD4+ and CD8+ T-lymphocyte toxicity, MICA-dependent complement fixation, and MICA-specific antibody-dependent cellular cytotoxicity.

33. A method of treating cancer comprising administering to a subject in need of treatment an effective amount of an opsonizing agent, wherein the opsonizing agent binds to MICA.

34. (canceled)

35. A method of identifying a subject who is a candidate for anti-MICA therapy comprising:
detecting evidence of overexpression of MICA in a sample from the subject as compared to expression of MICA in a control, wherein the evidence of overexpression of MICA is indicative of a subject who is a candidate for anti-MICA therapy.

36. A method of monitoring a course of treatment in a subject receiving anti-MICA therapy, the method comprising determining whether the level of sMICA in the subject after treatment comprises a reduced level of sMICA as compared to the level of sMICA in a control sample obtained from the subject at an earlier point in time, wherein the reduced level indicates that the anti-MICA therapy reduced the level of sMICA in the subject.

37. A method of monitoring an individual at risk for the progression of a pre-malignant plasma cell disorder, the method comprising:
a) providing a biological sample from the individual; and
b) determining the level of MICA or anti-MICA antibodies in the biological sample; and
c) comparing the measured level of MICA or anti-MICA antibodies with the level of MICA or anti-MICA antibodies in a control sample, wherein the presence of an altered level of MICA or anti-MICA antibodies in the individual's biological sample compared to the control sample indicates that the individual is at risk for progression of the pre-malignant plasma cell disorder.

38. (canceled)

39. The method of claim 37, wherein the MICA comprises soluble MICA or cell-associated MICA.

40. A method of treating a cancer, or a symptom of cancer or cancer progression in a subject, the method comprising administering to the subject an effective amount of an ERp5 modulating composition.

41. The method of claim 40, wherein the ERp5 modulating composition comprises an anti-ERp5 antibody.

42. A composition comprising a MICA modulating composition in a pharmaceutically acceptable carrier.

43. The composition of claim 42, wherein the MICA modulating composition comprises an anti-MICA antibody, an anti-protein disulfide isomerase (PDI) antibody or a combination thereof.

44-47. (canceled)

48. The composition of claim 42, further comprising an anti-CTLA-4 antibody.

49. The composition of claim 42, further comprising one or more tumor cell antigens that elicit an immune response against a tumor.

50. The composition of claim 42, wherein the tumor cell antigens include autologous tumor cells.

51. The composition of claim 50, wherein the autologous tumor cells express GM-CSF.

52. The composition of claim 42, further comprising an anti-CTLA-4 antibody and one or more tumor cell antigens that elicit an immune response against a tumor.

59. A composition comprising an ERp5 modulating composition in a pharmaceutically acceptable carrier.

60. The composition of claim 59, wherein the ERp5 modulating composition comprises an anti-ERp5 antibody.
